(19) United States
(12) Patent Application Publication
(10) Pub. No.: US 2015/0132775 A1
(43) Pub. Date: May 14, 2015

(54) METHOD AND DEVICE FOR DETERMINING CONCENTRATION, CROSSTALK AND DISPLACEMENT FLUORESCENCE CROSS CORRELATION SPECTROSCOPY

(71) Applicants: Johan Strömqvist, Stockholm (SE); Sofia Johansson, Sollentuna (SE); Jerker Widengren, Solna (SE)

(72) Inventors: Johan Strömqvist, Stockholm (SE); Sofia Johansson, Sollentuna (SE); Jerker Widengren, Solna (SE)

(73) Assignee: APICONISIS AB, Stockholm (SE)

(21) Appl. No.: 14/117,347

(22) PCT Filed: May 11, 2012
(86) PCT No.: PCT/SE2012/050504

§ 371 Date: Feb. 13, 2014

Related U.S. Application Data
(60) Provisional application No. 61/485,647, filed on May 13, 2011.

(51) Int. Cl. G01N 21/64; C12Q 1/68; G01N 33/58

(52) U.S. Cl. CPC G01N 21/6428; G01N 21/6408; G01N 33/582; C12Q 1/6818; G01N 2021/6417; G01N 2021/6441; G01N 2021/6432
USPC 435/7.23; 250/459.1; 250/458.1; 702/19; 436/501; 435/7.2

(57) ABSTRACT

The present invention provides a FCCS method for determining the concentration and/or the diffusion coefficient of at least a first labeled species, a second labeled species and/or a complex between said first and second labeled species, in a system, wherein the method comprises the steps of determining a cross-talk parameter K, wherein K is the ratio between the brightness of the first labeled species and the second labeled species at the centre of each focus, as detected for both species in the channel for detecting the second labeled species; using the cross talk parameter K for determining a displacement parameter rₒ and using K, rₒ, or both K and rₒ for determining the concentration and/or the diffusion coefficient of said first and/or a second labeled species and/or a complex between said first and second labeled species.

### TECHNICAL FIELD OF THE INVENTION

The present invention relates to a method, apparatus and computer program for spectroscopic measurement and analysis. The invention concerns Fluorescence Cross Correlation Spectroscopy (FCCS) to quantify concentrations and diffusion coefficients of reacting species and their products in membranes or in solutions. In addition, the invention also relates to FRET, where FCCS can be used to quantify FRET efficiencies.

### BACKGROUND ART

Fluorescence Correlation Spectroscopy (FCS) was developed in the 70's to monitor kinetics and molecular reactions in solutions^{1,2}. By forming a small confocal volume (femto litre) and correlating the detected fluorescence intensity of fluorescently labeled molecules, as they diffuse through the confocal volume, information regarding concentrations and diffusion coefficients can be extracted. FCCS generalize FCS to also include a second fluorescent dye (and sometimes also a second laser), emitting in a different spectral region^{3,4}. In this way one can easily distinguish two different species by their color and further cross-correlate the signal from the two different colors and thereby extract information about their interactions. FIG. 1 illustrates the confocal volumes of two lasers and two species labeled with two different fluorescent labels.

Well-known limitations of the FCCS technique are: 1) the cross-talk, which is the detection of e.g. green fluorescence in the detector intended to only detect e.g. red fluorescence; 2) the displacement, in case of two lasers, which is the unavoidable non-perfect overlap between the two excitation laser foci; 3) the uncertainty in the estimation of the “confocal radii” (see below for a more precise definition), ω_{G} and ω_{R}, in particular if the measurements are performed on a planar surface, for example a cell membrane. All of these parameters complicate the interpretation of FCCS data. Also additional quenching or FRET upon interaction complicates it even more.

Attempts to determine for example the displacement (not defined/termed above) has required extensive complicated equipment such as methods that utilize 2 focus FCS, which is a challenging technique to use and is time consuming. The ways to correct for crosstalk have had limitations in several aspects, both in terms of correctness and in terms of time; often a long series of control experiments have been required. FRET or quenching upon binding has been proposed to affect the FCCS curves; however, the other limiting parameters mentioned above makes these effects of FRET or quenching impossible to quantify.

To summarize, there is a need in the art for improved FCCS methods.

### SUMMARY OF THE INVENTION

It is an object of the present invention to provide an improvement of the prior art.

As a first aspect of the invention, there is provided a FCCS method for determining the concentration and/or the diffusion coefficient of at least a first labeled species, a second labeled species and/or a complex between the first and second labeled species, in a system, wherein the method comprises the steps of

a) determining a cross-talk parameter K, wherein K is the ratio between the brightness of the first labeled species and the second labeled species at the centre of each focus, as detected for both species in the channel for detecting the second labeled species;

b) using the cross talk parameter K for determining a displacement parameter rₒ, wherein r₀ is the displacement between the two lasers of the FCCS apparatus in the lateral dimension and

c) using K, rₒ, or both K and rₒ for determining the concentration and/or the diffusion coefficient of the first and/or a second labeled species and/or a complex between said first and second labeled species.

FCCS refers to fluorescence cross correlation spectroscopy.

A FCCS method is thus a method that uses information, such as data and measured correlation functions, obtained from a FCCS apparatus. A FCCS apparatus may thus perform all or some of the method steps. As an example, steps a) and b) b may be performed with the use of a FCCS apparatus.

The method of the first aspect of the invention may thus be performed by a FCCS apparatus comprising a first laser for exciting the first labeled species, the same or a second laser for exciting the second labeled species as well as a first channel for detecting fluorescence from the first labeled species and a second channel for detecting fluorescence from the second labeled species.

Thus, as a configuration of the first aspect of the invention, there is provided a method for determining the concentration and/or the diffusion coefficient of at least a first labeled species, a second labeled species and/or a complex between the first and second labeled species, in a system, by the use of a FCCS apparatus, wherein the FCCS apparatus comprises a first laser for exciting the first labeled species, the same or a second laser for exciting the second labeled species as well as a first channel for detecting fluorescence from the first labeled species and a second channel for detecting fluorescence from the second labeled species;

wherein the method comprises the steps of

a) determining a cross-talk parameter K, wherein K is the ratio between the brightness of the first labeled species and the second labeled species at the centre of each focus of the first and second lasers, as detected for both labeled species in the second channel for detecting the second labeled species;

b) using the cross talk parameter K for determining a displacement parameter rₒ, wherein r₀ is the displacement between the two lasers of the FCCS apparatus in the lateral dimension, and

c) using K, rₒ, or both K and rₒ for determining the concentration and/or the diffusion coefficient of the first and/or a second labeled species and/or a complex between the first and second labeled species.

In other words, step b) may comprise determining a displacement parameter rₒ by using the cross-talk parameter K determined from step a).

It is to be understood that step b) may also include specifying that K is negligible, i.e. that K is set to zero. Thus, step a) may lead to determining that K is negligible. This may for example be a case if more than one laser is used in the FCCS apparatus. In systems with only one laser, there is usually more cross-talk present.

Further, in other words, step c) may comprise determining the concentration and/or the diffusion coefficient of the first and/or a second labeled species and/or a complex between the first and second labeled species by using the cross-talk parameter K determined from step a and the displacement parameter rₒ determined from step b). Step c) may thus comprise only using K, only using rₒ, or using both K and rₒ.

The “system” may be a single cell, and may comprise the membrane of a single cell.

The first aspect of the invention is based on the inventors insight that a cross-talk parameter K, which is the ratio between the brightness of the first labeled species and the second labeled species at the centre of each focus, as detected for both species in the channel for detecting the second labeled species, may be determined, which provides for more accurate determination of different parameters of a studied system. The invention provides for calculation of the displacement between the excitation foci of the two lasers, in case of dual-color FCCS. The invention also provides for correction for crosstalk in a new and more accurate manner and determination of the distorted ω_{G} and ω_{R}, the radii of the fluorescence brightness distributions, such as the red and green fluorescence brightness distributions, respectively, on a membrane, or other surfaces. Here, radius is defined as the distance from the maximal fluorescence brightness distribution to where it has dropped with a factor e². The invention also provides for determination of the FRET efficiency upon binding and determination of the concentration and diffusion coefficients of involved labeled species in a studied system. The invention can further be used to quantify for example the strength of protein-protein interactions (PPI:s) and characterize compounds modulating PPI:s. The method of the first aspect may be used for determining concentrations of labeled species in single cells, such as in the cell membrane of single cells, with high accuracy.

The method according to the present invention may further comprise the initial steps of providing at least one sample comprising a first and a second labeled species. The sample may thus comprise the system to be analysed.

The sample may be provided in the FCCS apparatus.

The labeled species may be fluorescently labeled species. The fluorescent label may be an external label, such as a fluorophore, or an intrinsic label, such as with a GFP (green fluorescent protein)-labeled species.

Consequently, in embodiments of the first aspect of the invention, the method is further comprising the initial step a₀) of providing at least one sample comprising the system to be analysed in an FCCS apparatus and obtaining the cross correlation function G_{RT}(T) of the first and second labeled species and the autocorrelation functions, G_{R}(T) and G_{T}(T), of the first and second labeled species.

The skilled person understands how to obtain the autocorrelation functions and the cross correlation function from a sample using FCCS. Further, step c) may comprise fitting at least one of the correlation functions obtained in step a₀ to Equation 4c of the disclosure and using the determined K and rₒ in Equation 4c for determining the concentration and/or the diffusion coefficient of the first and/or a second labeled species and/or a complex between said first and second labeled species.

In embodiments of the first aspect of the invention, the FCCS apparatus comprises a first laser for exciting the first labeled species, the same or a second laser for exciting the second labeled species as well as a first channel for detecting fluorescence from the first labeled species and a second channel for detecting fluorescence from the second labeled species.

It is further to be understood that a FCCs-apparatus of the present disclosure may comprise more than two lasers and/or more than two detectors, i.e. more than two channels for detecting fluorescence.

The system may be a single cell. The method may thus be used for determining the concentration and/or diffusion coefficient of e.g. membrane proteins of single cells.

The system may further comprise a population of single cells and the method may thus be used for determining the average diffusion or concentration of the population of cells, such as in the membrane of the cells.

As discussed above, the first and/or second labeled species may be fluorescently labeled species.

The first and second species may be fluorescent species emitting in different or only partly overlapping, spectral regions.

In embodiments of the first and second aspect, the fluorescence emission of the first labeled species is blue shifted with respect to the fluorescence emission from the second species and the channels for detecting each of the labeled species are suitable for their respective spectral range of their fluorescence.

Thus, the first labeled species may emit light in the green region and the second labeled species may emit light in the red region of the spectra. The green region may for example be defined as between about 490-560 nm and the red region may for example be defined as between about 635-700 nm.

Consequently, the two species may for example be fluorescent labeled species, in which one is labeled with a green fluorophore and another labeled with a red fluorophore.

In an embodiment of the first aspect, the first labeled species is a green fluorescent species and the second species is a red fluorescent species and the channel for detecting the second labeled species is suitable for detecting red fluorescence.

K may be defined as the ratio between the brightness times the excitation cross-section of the first labeled species and the second labeled species at the centre of each focus, as detected for both species in the channel for detecting the second labeled species. Thus, K may be proportional to the ratio between the brightness of the first labeled species and the second labeled species at the centre of each focus, as detected for both species in the channel for detecting the second labeled species. If the excitation cross-section of the first and the second species are similar, then in embodiments K may be defined as

$$K = \frac{{W}_{max}^{G} {κ}_{g}^{R} {q}_{g}}{{W}_{max}^{R} {κ}_{r}^{R} {q}_{r}}$$

wherein the parameters are as defined in the description of the invention.

Further, if the excitation cross-section of the first and the second species are different, then K may also be defined as

$$K = \frac{{W}_{max}^{G} {κ}_{g}^{R} {q}_{g} {σ}_{g}}{{W}_{max}^{R} {κ}_{r}^{R} {q}_{r} {σ}_{r}}$$

in which σ_{g} is the excitation cross section of the first labeled species and σᵣ is the excitation cross section of the second labeled species.

The brightness may be at the center of each laser foci.

Wₘₐₓ^{G} refers to the maximal value of the brightness distribution, W_{G}( r̅) of the first labeled species. The brightness distribution is defined as the product of the excitation intensity (for the laser exciting the first labelled species), I_{exc}( r̅), and the collection efficiency function, CEF( r̅).

κ_{g}^{R} refers to the detection efficiency of the first labeled species in the detector intended to detect the second labeled species.

q_{g} refers to the quantum yield of the first labeled species.

Wₘₐₓ^{R} refers to the maximal value of the brightness distribution, W_{R} ( r̅), of the second labeled species. The brightness distribution is defined as the excitation intensity (for the laser exciting the second labeled species), I_{exc}( r̅), and the collection efficiency function, CEF( r̅).

κᵣ^{R} refers to the detection efficiency of the second labeled species in the detector intended to detect the second labeled species.

qᵣ refers to the quantum yield of the second labeled species.

r₀, i.e. the displacement between the two lasers of the FCCS apparatus in the lateral dimension, may be defined as

\sqrt{x₀²+y₀²}

in which x and y are in the lateral and axial dimensions.

Thus, if for example the first labeled species is a green fluorescent species and the second labeled species is a red fluorescent species, then

Wₘₐₓ^{G} refers to the maximal value of the green fluorescence brightness distribution, W_{G}( r̅), defined as the product of the excitation intensity (for the laser exciting the green species), I_{exc}( r̅), and the collection efficiency function, CEF( r̅)

κ_{g}^{R} refers to the detection efficiency of the green species in the detector intended to detect red fluorescence.

q_{g} refers to the quantum yield of the green fluorescent species.

σ_{g} refers to the excitation cross section of the green fluorescent species.

Wₘₐₓ^{R} refers to the maximal value of the red fluorescence brightness distribution, W_{R}( r̅), defined as the excitation intensity (for the laser exciting the red species), I_{exc}( r̅), and the collection efficiency function, CEF ( r̅)·κᵣ^{R} refers to the detection efficiency of the red species in the detector intended to detect red fluorescence

qᵣ refers to the quantum yield of the red fluorescent species

σᵣ refers to the excitation cross section of the red fluorescent species.

In an embodiment of the first aspect, step a) comprises determining K via a negative control, in which two species lacking mutual interactions are utilized.

The skilled person understands how to determine K from such a control from the information given herein. As an example, a negative control may be performed by adding a red fluorescently labeled antibody, specific to a membrane protein A, to cells which contain membrane protein A and have a membrane protein B intrinsically green labeled, wherein A and B are known not to interact. FCCS measurements may then be performed on these cells. From the FCCS data, the parameter K can be determined.

In an embodiment of the first aspect, step b) comprises performing FCCS measurements on two species that interact with each other (a positive control). Interaction may be the binding of one species to the other.

As an example, a positive control may be performed by adding a red fluorescently labeled antibody, specific to a membrane protein B, to cells which contain membrane protein B intrinsically green labeled. Then perform FCCS measurements on these cells. From the FCCS data, the parameter r₀ can be determined if K has been determined from the negative control and the brightness ratio between the labeled antibody specific to A and the labeled antibody specific to B is known.

Step c) may also comprise determination of z₀, ω_{G}, ω_{R}, z_{G}, and z_{R},

z₀ denotes the displacement between the two lasers of the FCCS apparatus in the axial dimension and wherein the radial distances from the maximum point of W_{G}( r̅) and W_{R}( r̅) to where they have dropped by a factor of e² is denoted ω_{G} and ω_{R} in the lateral direction and z_{G} and z_{R} in the axial direction, respectively and

wherein W_{G}( r̅) is the detected fluorescence brightness distribution of the first labelled species and

W_{R}( r̅) is the detected fluorescence brightness distribution of the second labelled species.

Consequently, in an embodiment of the first aspect, step c) comprises

c1) determining z₀, ω_{G}, ω_{R}, z_{G}, and z_{R}, and

c2) utilizing Equation 4c as disclosed herein for determining the concentration and/or the diffusion coefficient of the first and/or second species.

If z₀ is determined at step c) then it may not be necessary to determine ro in step b), i.e. the method may exclude step b).

In an embodiment of the first aspect, step c2) comprises performing an FCCS experiment and fitting the three measured correlation curves to Eq. 4C.

The fitting of curves in the present disclosure may be performed by letting all known variables be free to vary. Further, the fitting may also be performed by approximating one or several parameter values that is being fixed during fitting.

The measured correlation curves are thus the autocorrelation curves and the cross correlation curves

Fitting may for example comprise maximum entropy or non-linear least squares optimization routines.

As an example, the step of fitting the autocorrelation curves and the cross correlation curves may be performed simultaneously. This may be a global fit in which some of the parameters are common between the fits. This may be more advantageous in that it provides for a more accurate estimation of relevant parameters for the system.

In an embodiment of the first aspect, there is FRET between the first and second species, and step c) comprises

c1) determining z₀, ω_{G}, ω_{R}, z_{G}, and z_{R}, and

c2) utilizing Equation 6 as disclosed herein for determining the concentration and/or the diffusion coefficient of the first and/or second species and/or a complex between said first and second labeled species.

FRET refers to fluorescence or Förster resonance energy transfer and is known to a person skilled in the art⁵.

As an example, step c2) comprises performing an FCCS experiment and fitting the autocorrelation curves and the cross correlation curves to Eq. 4C in combination with the substitutions mentioned in Section 4.5. In embodiments of the first aspect, the first and second species are labeled DNA strands.

In embodiments of the first aspect, the first species is a labeled binding agent and the second species is labeled membrane protein, or vice versa.

The binding agent may for example be an antibody. The label may be a fluorescent label. Thus, this allows for studies of membrane proteins and interactions between membrane proteins.

Further, both the first and second species may be labeled binding agents or labeled proteins, such as labeled membrane proteins.

As an example, the labeled membrane protein is in a cell membrane. This thus allows for live cell studies of membrane proteins.

As a second aspect of the invention, there is provided a method for determining the equilibrium constant K_{D} in the reaction between a first and second species, wherein

$${K}_{D} = \frac{⌊ {A}_{free} ⌋ \cdot ⌊ {B}_{free} ⌋}{[ AB ]}$$

in which [A_{free}] is the concentration of unbound first species, [B_{free}] is the concentration of unbound second species and [AB] is the concentration of the complex between A and B, the method comprising

a3) providing at least one sample comprising a labeled first species and a labeled second species,;

b3) measuring the concentration of the first and the second labeled species and the complex between the first and second labeled species in the sample according to the method according to the method according to the first aspect of the invention,

c3) determining K_{D} from the measured concentrations of step b1.

The terms and definitions used in relation to the other objects also apply to this aspect of the invention.

Thus, due to the high accuracy concentration data obtained by the method according to the invention, the present invention provides for determination of K_{D} with high accuracy.

It is to be understood that other constants, such as other constants related to the affinity, binding strength or equilibrium between A and B, may be used instead of K_{D} as defined above.

In embodiments, step a3) comprises providing at least two samples, wherein the samples have different concentrations of labeled first and second species A and B, and step b3) comprises measuring the concentration of the first and the second labeled species and the complex between the first and second labeled species in each sample.

In embodiments, the determination of K_{D} in step c3) comprises fitting the measured concentrations to

$$γ = \frac{[ AB ]}{[ A ]} = \frac{{K}_{D} + [ A ] + [ B ] + \sqrt{{( {K}_{D} + [ A ] + [ B ] )}^{2} - 4 \cdot [ A ] \cdot [ B ]}}{2 [ A ]} ,$$

wherein γ is the fraction of bound first species, and [A]=└A_{free}┘+[AB] and [B]=└B_{free}┘+[AB] are the total concentrations of the first and second labelled species, respectively, in which [AB] is the concentration of the complex between the first and second labelled species.

Further, each sample may be a single cell. Thus, step a3 may comprise providing a system with different cells and step b3 may thus comprise measuring the concentration on each single cell. Any or both of the first and second species may be bound to the cell membrane of the cells. In embodiments, both the first and/or second species are membrane proteins.

As a third aspect of the invention, there is provided a method for determining if a compound P promotes or inhibits the interaction between a first and second species A and B, comprising measuring the concentration of the first and the second labeled species and the complex between the first and second labeled species in at least one sample according to the method according to the first aspect of the invention, and

analyzing how [AB] as a function of [A] and/or [B] varies in the presence of compound P.

The terms and definitions used in relation to the other objects also apply to this aspect of the invention.

The inventors have found that by determining concentrations of species and complex between species (A and B) according to the method of the present invention and studying how the presence of compound P affects an equilibrium constant, e.g. K_{D}, in a reaction between A and B, it provides for determining if compound P promotes or inhibits the interaction between a first and second species A and B. The reaction may for example be a diffusion-limited bimolecular reaction between A and B. For example, this may be performed by generating plots of [AB] as a function of [A] and/or [B] and studying how the presence of a compound P affects the plot. Compound P may thus be a drug and the method thus allows for determining if a drug may modulate e.g. receptor-receptor interactions in a cell membrane. Compound P may thus be a small organic molecule and A and B may be proteins, such as receptor proteins or other proteins in the cell membrane.

In principle, only one sample may be analysed, such as a single cell.

However, at least two samples may be analysed, such as at least ten samples, such as at least 30 samples, such as at least 50 samples. This means, if the samples are single cells, then at least two cells may be analysed, such as at least ten cells, such as at least 30 cells, such as at least 50 cells.

A and B may for example be proteins, such as membrane proteins of a cell.

In embodiments, the method is comprising

a4) providing at least one sample comprising a labeled first species A and a labeled second species B;

b4) measuring the concentration of the first and the second labeled species and the complex between the first and second labeled species in the sample according to the method according to the method of the first aspect of the invention;,

c4) providing at least one sample comprising the labeled first species and the labeled second species and the compound P;

d4) measuring the concentration of the first and the second labeled species and the complex between the first and second labeled species in the sample of c4) according to the method according to the first aspect of the invention;

e4) generating plot P1 of [AB] as a function of [A] and/or [B] for the data obtained in step b2) and plot P2 of [AB] as a function of [A] and/or [B] for the data obtained in step d2); wherein [A]=└A_{free}+[AB] and [B]=└B_{free}+[AB] are the total concentrations of the first and second labelled species, respectively, in which [AB] is the concentration of the complex between the first and second labelled species; and

f4) determining that compound P promotes interaction between A and B if plot P2 is shifted towards the [AB]-axis as compared to plot P1 or determining that compound P inhibits interaction between A and B if plot P2 is shifted away from the [AB]-axis as compared to plot P1.

In other words, step f4) may comprise determining that compound P promotes interaction between A and B if plot P2 is shifted along the [A] or [B] axis towards lower parameter values as compared to plot P1 or determining that compound P inhibits interaction between A and B if plot P2 is shifted along the [A] or [B] axis towards higher parameter values as compared to plot P1

It is to be understood that plots P1 and P2 are similar plots i.e. if plot P1 is a plot of [AB] as a function of [A], then also P2 is a plot of [AB] as a function of [A]. Thus, P1 may be a plot of [AB] as a function of [A] when P2 is a plot of [AB] as a function of [A]. Further, P1 may be a plot of [AB] as a function of [B] when P2 is a plot of [AB] as a function of [B]. Moreover, P1 may be a plot of [AB] as a function of [A] and [B] when P2 is a plot of [AB] as a function of [A] and [B]

Further, it is to be understood that if [AB] is plotted on the Y-axis and for example [A] or [B] is plotted on the x-axis (as in FIG. 7 of the present disclosure), then compound P promotes interaction between A and B if plot P2 is shifted towards lower parameter values as compared to plot P1 and compound P inhibits interaction between A and B if plot P2 is towards higher parameter values as compared to plot P1

For example, step a4) may comprise providing at least two samples, wherein the samples have different concentrations of labeled first and second species A and B, and wherein step b4) comprises measuring the concentration of the first and the second labeled species and the complex between the first and second labeled species in each sample.

In analogy, step c4 may comprise providing at least two samples, wherein the samples have different concentrations of labeled first and second species A and B, and wherein step d4) comprises measuring the concentration of the first and the second labeled species and the complex between the first and second labeled species in each sample.

Furthermore, the samples of step a4) and c4) may be the same samples. This means that at least one sample may be provided in step a4) and compound P is added to the sample at step d4).

In embodiments, step e4) comprises determination of K_{D} for the samples of steps a4) and c4) by fitting the measured concentrations to

$$γ = \frac{[ AB ]}{[ A ]} = \frac{{K}_{D} + [ A ] + [ B ] + \sqrt{{( {K}_{D} + [ A ] + [ B ] )}^{2} - 4 \cdot [ A ] \cdot [ B ]}}{2 [ A ]} ,$$

wherein γ is the fraction of bound first species, and [A]=└A_{free}┘[AB] and [B]=└B_{free}┘+[AB] are the total concentrations of the first and second labelled species, respectively, in which [AB] is the concentration of the complex between the first and second labelled species,
and wherein plot P1 is generated by using the determined K_{D} and the average [A] and/or [B] for the samples of step a4) and plot P2 is generated by using the determined K_{D} and the average [A] and/or [B] for the samples of step c4).

It is to be understood that other constants, such as other constants related to the affinity, binding strength or equilibrium between A and B, may be used instead of K_{D} as defined above.

In embodiments of the third aspect, step e4) does not require the generation of plots. For example, the method may comprise determining K_{D} in the absence and presence of compound P, with or without generating plots P1 and P2, and then, such as in step f4), determining that compound P promotes interaction between A and B if K_{D} is lower in the presence of compound P or determining that compound P inhibits interaction between A and B if K_{D} is higher in the presence of compound P.

As discussed above each sample may be a single cell. Further the first and second species may be bound to the cell membrane of the single cells.

Accordingly, each sample may be a single cell and the first labeled species may be a labeled membrane protein that is endogenously expressed by said cell, and the second species may be a transfected labeled membrane protein.

An endogenously expressed protein may refer to a protein that is “naturally” expressed by the cell. A transfected labeled protein refers to a protein that is expressed in the cell due to transfection of the cell and that is intrinsically labeled, e.g. GFP-labeled.

Thus, since transfected proteins usually are expressed in different amounts by different individual, or single, cells, different concentrations of this protein is obtained in a population of single cells. The endogenous receptor is however usually expressed in similar amounts in the population of cells. This allows or facilitates the generation of the plots discussed above and thereby the determination of whether compound P promotes or inhibits the interaction between a first and second species A and B.

In other words, out of the two interacting proteins, at least one of them may differ in concentration between different single cells. This may be due to the endogenous genetical or posttranscriptional control, or how the genetic construct of a transgenic protein is generated. The expression level of endogenous proteins may also be experimentally modulated, for instance by partially inhibiting the expression with micro-RNA.

As a fourth aspect of the invention, there is provided a method for calculating the displacement r₀ between the excitation foci of two lasers, comprising the steps of

a) determining a cross-talk parameter K, wherein K is the ratio between the brightness of a first labeled species and a second labeled species at the centre of each focus, as detected for both species in a channel for detecting the second labeled species;

b) using the cross talk parameter K for determining the displacement parameter rₒ.

The terms and definitions used in relation to the other objects also applies to this aspect of the invention.

The fourth aspect provides for determination of the displacement radius between two focused laser beams, which is advantageous in that it provides for more accurate measurements using the lasers, such as in FCCS-measurements.

In an embodiment of the fourth aspect, the method is performed in a FCCS system.

In an embodiment of the fourth aspect, the first labeled species is fluorescent in the spectral region of about 490-560 nm, and the second species is fluorescent in the spectral region of about 635-700 nm and the channel for detecting the second labeled species is suitable for detecting fluorescence of about 635-700 nm. Thus, the first labelled species may be a green fluorescent species and the second labelled species may be a red fluorescent species.

Of course, other fluorescent species may be used that emit in different or only partly overlapping, spectral regions.

In embodiments of the fourth aspect, K is defined as in relation to the first aspect above.

Thus, in embodiments of the second aspect K may be defined as

$$K = \frac{{W}_{ma x}^{G} {κ}_{g}^{R} {q}_{g}}{{W}_{m ax}^{R} {κ}_{r}^{R} {q}_{r}}$$

if the excitation cross-section of the first and the second species are similar and wherein the parameters are as defined in the description of the invention.

Further, as discussed above, if the excitation cross-section of the first and the second species are different, then K may also be defined as

$$K = \frac{{W}_{ma x}^{G} {κ}_{g}^{R} {q}_{g} {σ}_{g}}{{W}_{ma x}^{R} {κ}_{r}^{R} {q}_{r} {σ}_{r}}$$

in which σ_{g} is the excitation cross section of the first labeled species and σᵣ is the excitation cross section of the second labeled species

In an embodiment of the fourth aspect, step a) comprises determining K via a negative control, in which two species lacking mutual interactions are utilized.

In an embodiment of the fourth aspect, step b) comprises performing FCCS measurements on two species that interact with each other (a positive control).

A negative and positive control may be performed as described in relation to the first aspect above.

As a fifth aspect of the invention, there is provided a FCCS device for determining the concentration and/or the diffusion coefficient of at least a first labeled species, a second labeled species and/or a complex between the first and second labeled species, the device comprising
a FCCS apparatus comprising a first laser for exciting the first labeled species, a second laser for exciting the second labeled species, a first channel for detecting fluorescence from the first labeled species and a second channel for detecting fluorescence from the second labeled species
an estimation unit adapted to
   determine a cross-talk parameter K, wherein K is proportional to the ratio between the brightness of the first labeled species and the second labeled species at the centre of each focus, as detected for both species in the channel for detecting the second labeled species;
   determine a displacement parameter rₒ, wherein r₀ is the displacement between the two lasers of the FCCS apparatus in the lateral dimension, by the use of the determined the cross talk parameter K, and
   determine the concentration and/or the diffusion coefficient of the first and/or a second labeled species by the use of both the determined K and rₒ.

The terms and definitions used in relation to the other objects also applies to this aspect of the invention.

The estimation unit may for example comprise or be constituted by a processing unit or a computer adapted to access a local or remotely located database or the like by means of wired or wireless communication techniques known in the art, The estimation unit may comprise software or have access to software for determining K, rₒ and the concentration and/or the diffusion coefficient of the first and/or a second labeled species and/or a complex between said first and second labeled species.

In embodiments, the estimation unit is further adapted to obtain the cross correlation function G_{RT}(T) of the first and second labeled species and the autocorrelation functions, G_{R}(T) and G_{T}(T), of the first and second labeled species. The estimation unit may thus be adapted also to display the cross correlation function and the autocorrelation functions on a screen.

Further, the estimation unit may further be adapted to fit at least one of the correlation functions obtained to Equation 4c of the disclosure and use the determined K and r_{c}, in Equation 4c for determining the concentration and/or the diffusion coefficient of the first and/or a second labeled species and/or a complex between said first and second labeled species. The estimation unit may comprise software or have access to software for fitting at least one of the correlation functions obtained to Equation 4c of the disclosure. For example, the estimation unit may be adapted to fit the autocorrelation curves and the cross correlation curves to Eq. 4C.

In embodiments, the estimation unit is further adapted to determine z₀, ω_{G}, ω_{R}, z_{G}, and z_{R}, and utilizing Equation 4c as disclosed herein for determining the concentration and/or the diffusion coefficient of the first and/or second species, wherein z₀ denotes the displacement between the two lasers of the FCCS apparatus in the axial dimension and wherein the radial distances from the maximum point of W_{G} ( r̅) and ω_{R}( r̅) to where they have dropped by a factor of e² is denoted ω_{G} and ω_{R} in the lateral direction and z_{G} and z_{R} in the axial direction, respectively and

wherein W_{G}( r̅) is the detected fluorescence brightness distribution of the first labelled species and

W_{R}( r̅) is the detected fluorescence brightness distribution of the second labelled species.

It is also understood that the estimation unit may be adapted to perform any of the method steps as disclosed in relation to the second and third aspect discussed above.

As a sixth aspect of the invention, there is provided the use of a cross-talk parameter K, wherein K is the ratio between the brightness of a first labeled species and a second labeled species at the centre of two laser foci as detected for both species in the channel for detecting the second labeled species, for fitting experimental data from a FCCS experiment to at least one correlation function or cross correlation function.

The correlation function may thus be the correlation function of the first or second labeled species. The cross correlation function may be the cross correlation function that relates to the interaction between the first and second labeled species.

As a seventh aspect of the invention, there is provided computer program product comprising computer-executable components for causing a device to perform any one or all of the steps recited in any of the invention when the computer-executable components are run on a processing unit included in the device.

The computer program product may thus be software and may perform any of the steps described in relation to any aspect of the invention. As an example, the computer program product may fit experimental data to correlation functions and estimate parameters of correlation functions.

The invention is thus a framework of methods, apparatus and computer programs to alleviate the above-mentioned problems of the prior art. By utilizing this framework, quantitative information regarding concentrations and diffusion coefficients of all the involved species can be determined. In addition, the FRET efficiency upon binding can be quantified.

### BRIEF DESCRIPTION OF THE DRAWINGS

FIG. 1 shows two different species 5 and 6, fluorescently labeled (3 and 4, respectively) diffusing in a membrane 7. Two different foci, 1 and 2, of different laser wavelength are forming the confocal volumes perpendicular to the membrane 7. Notice that the center of each focus is not overlapping. Hence, the displacement parameter r₀ does not vanish.

FIG. 2 shows the three correlation curves, 7, 8 and 9, of an FCCS experiment. The red 7 and the green 8 curves are the autocorrelation curves of the red and the green species, respectively, and the orange curve 9 is the cross-correlation curve. The black lines are fitted curves based on Eq. 4C.

FIG. 3 shows: Left: Two complementary single strands 12 labeled with two fluorescent dyes, 10 and 11, moving freely to each other. Right: same strands but hybridized

FIG. 4 shows: Left: An intrinsically labeled (with fluorescent protein 15) membrane protein 13 and the non-interacting protein 14, fluorescently labeled (with fluorescent dye 17) via an antibody 16, in the membrane 19 of a cell. Right: The same protein 13, now labeled (with fluorescent dye 17) via antibody 18 bound to it.

FIG. 5 shows: Left: the non-interacting labeled proteins 22 and 23. Right: the interacting proteins 23 and 24. Upon interaction, the distance between the two fluorescent labels, 20 and 21, is within the FRET range.

FIG. 6 shows: An intrinsically labeled (with fluorescent protein 27) membrane protein 25 and a endogenously expressed protein 26, fluorescently labeled (with fluorescent dye 29) via an antibody 28, in the membrane of a cell. The modulating compound 30 either promote the interaction of protein 25 with protein 26 or block the interaction.

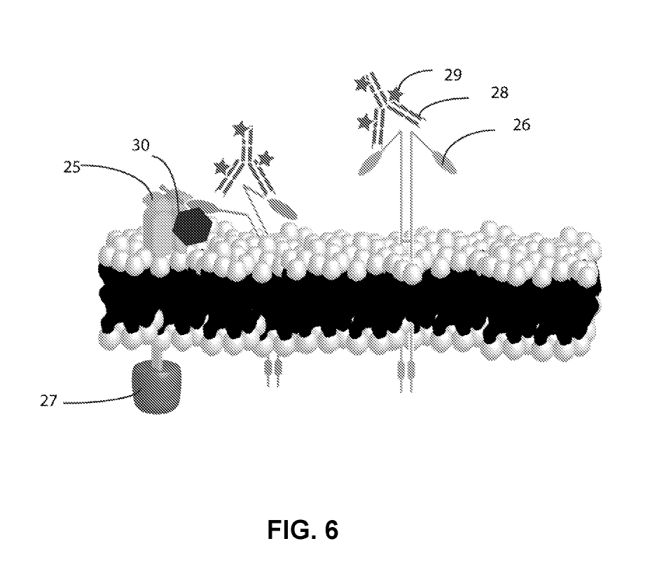

FIG. 7 shows: The percentage of endogenously expressed protein 26 that is bound to the transfected protein 25. Each dot represents an individual cell. Arrow 31 indicate a shift of the curve, towards the left, as the modulating compound 30 promotes the interaction of protein 25 with protein 26, while arrow 32 indicate a curve shift towards the right if modulator 26 blocks the interaction.

FIG. 8 shows: Typical auto- and cross-correlation curves of D^{d}-EGFP with: D^{d}-ab (left column), K^{b}-ab (middle column), and Ly49A-ab (right column). Green squares represent auto-correlation curves from D^{d}-EGFPs in all plots. Red circles show autocorrelation curves from Al647-labelled antibodies against: H-2D^{d} (left), H-2K^{b} (middle), and Ly49A (left). Orange triangles represent the corresponding cross-correlation curves between D^{d}-EGFPs and these three species. Black lines represent the corresponding fits, according to Eq. 4C. Each row contains three different concentration ratios between receptor and ligand (except for the positive control, which only contains two different ratios), from top to bottom: 0.3 and 0.07 (left); 1.3, 0.3 and 0.05 (middle); and 1.8, 0.2 and 0.05 (right).

### DETAILED DESCRIPTION OF THE INVENTION

The invention takes advantage of a refined and modified FCCS model, which is a mathematical expression that describes how parameters such as concentrations, diffusion coefficients, displacement, the focal volume and crosstalk are related to the experimental FCCS curves. This relationship will be derived in section 4.1. In section 4.2 to 4.5, the general ideas of how to determine all relevant parameters are explained. In section 4.6 to 4.7 we give examples of test systems that could be employed to find the parameters of interest. Finally, in section 4.8 we provide an application.

### 4.1 FCCS Theory

In FCS measurements, fluctuations in the detected fluorescence intensity, F(t), are typically generated as molecules diffuse in and out of a focused laser beam. These fluctuations, ∂F(t), are auto-correlated according to:

$$G ( τ ) = \frac{〈 ( F ( t ) - 〈 F ( t ) 〉 ) ( F ( t + τ ) - 〈 F ( t ) 〉 ) 〉}{{〈 F ( t ) 〉}^{2}} = \frac{〈 \partial F ( t ) \partial F ( t + τ ) 〉}{{〈 F ( t ) 〉}^{2}} , \qquad ( 1 )$$

here brackets denote time average.

For molecules undergoing diffusion in a volume, the detected intensity fluctuations, originating from the concentration fluctuations, ∂c( r̅,t), of a certain species at time t, is given by:

∂F(t)=κq∫_{R}_{²}W( r̅)∂c( r̅,t)∂²r (2)

Here κ denote detection efficiency, q fluorescence quantum yield of the species and W( r̅)=CEF( r̅)I_{exc}( r̅) is the detected fluorescence brightness distribution, a product of the excitation intensity I_{exc}( r̅) and the collection efficiency function CEF( r̅).

For interaction studies between two species labelled with different fluorophores, emitting in a green (G) and a red (R) spectral range, the fluorescence fluctuations in the G and R range may be cross-correlated according to:

$$\begin{matrix}{G}_{GR} ( τ ) = \frac{〈 ( {F}_{G} ( t ) - 〈 {F}_{G} ( t ) 〉 ) ( {F}_{R} ( t + τ ) - 〈 {F}_{R} ( t ) 〉 ) 〉}{〈 {F}_{G} ( t ) 〉 〈 {F}_{R} ( t ) 〉} \\ = \frac{〈 \partial {F}_{G} ( t ) \partial {F}_{R} ( t + τ ) 〉}{〈 {F}_{G} ( t ) 〉 〈 {F}_{R} ( t ) 〉}\end{matrix} \qquad ( 3 )$$

When the following assumptions hold: 1) W( r̅) has a Gaussian distribution, 2) the expectation value, <F(t)>, is time-independent, 3) the quantum yield of the green and the red species are unaffected upon binding and 4) only diffusion causes the fluctuations, then the cross-correlation and the autocorrelation functions of the fluorescence in G and R have the following analytical expressions⁶:

$$\begin{matrix}\{ \begin{matrix}{G}_{GR} ( τ ) - 1 = \frac{{c}_{g r} {Diff}_{g r}^{GR} ( τ )}{{V}_{GR} ( {c}_{g} + {c}_{g r} ) ( {c}_{r} + {c}_{g r} )} \\ {G}_{R} ( τ ) - 1 = \frac{{c}_{r} {Diff}_{r}^{R} ( τ ) + {c}_{g r} {Diff}_{g r}^{G} ( τ )}{{{V}_{G} ( {c}_{g} + {c}_{g r} )}^{2}} \\ {G}_{G} ( τ ) - 1 = \frac{{c}_{g} {Diff}_{g}^{G} ( τ ) + {c}_{g r} {Diff}_{g r}^{G} ( τ )}{{{V}_{G} ( {c}_{g} + {c}_{ g r} )}^{2}}\end{matrix} & ( 4 A ) \\ \{ \begin{matrix}{Diff}_{u}^{G} ( τ ) = {( 1 + \frac{4 {D}_{u} τ}{{ω}_{G}^{2}} )}^{- 1} {( 1 + \frac{4 {D}_{u} τ}{{z}_{G}^{2}} )}^{- 1 / 2} \\ {Diff}_{u}^{R} ( τ ) = {( 1 + \frac{4 {D}_{u} τ}{{ω}_{R}^{2}} )}^{- 1} {( 1 + \frac{4 {D}_{u} τ}{{z}_{R}^{2}} )}^{- 1 / 2} \\ {Diff}_{g r}^{GR} ( τ ) = {( 1 + \frac{4 {D}_{g r} τ}{( {ω}_{G}^{2} + {ω}_{R}^{2} ) / 2} )}^{- 1} {( 1 + \frac{4 {D}_{g r} τ}{( {z}_{G}^{2} + {z}_{R}^{2} ) / 2} )}^{- 1 / 2}\end{matrix} & ( 4 B )\end{matrix}$$

Here, c_{g}, cᵣ and c_{gr} are the concentrations of the two free species and their complex, respectively. The subscript u denotes the green (g), the red (r) or the red-and-green (gr) emitting species, and Dᵤ denotes their corresponding diffusion coefficients.

The radial distances from the maximum point of W_{G}( r̅) and W_{R}( r̅) to where they have dropped by a factor of e² is denoted ω_{G} and ω_{R} in the lateral direction and z_{G} and z_{R} in the axial direction, respectively.

V_{G}=(∫W_{G}( r̅)∂³r)²/∫W_{G} ( r̅)²∂³r and V_{R}=(∫W_{R}( r̅)∂³r)²/∫W_{R}( r̅)²∂³r are the effective detection areas of the green and red laser foci, and V_{GR} is the corresponding green-red detection volume.
(V_{GR}=(∫W_{G}( r̅)W_{R}( r̅′)∂³r∂³r′)/∫W_{G}( r̅)W_{G}( r̅)W_{R}( r̅)d³r) when the focal volumes overlap perfectly. However, in FCCS measurements based on excitation from two lasers, the focal overlap is typically not perfect. Moreover, considerations such as cross-talk from the green dye into the red channel and background fluorescence need also be taken into account (appendixes section). This leads to the following Equations denoted as 4C:

$$\{ \begin{matrix}{G}_{GR} ( τ ) - 1 = \frac{\begin{matrix}\frac{{V}_{G} {V}_{R}}{{V}_{GR}} {c}_{g r} {e}^{- ( \frac{2 {r}_{0}^{2}}{{ω}_{G}^{2} + {ω}_{R}^{2} + 8 {D}_{g r} τ} + \frac{2 {z}_{0}^{2}}{{z}_{G}^{2} + {z}_{R}^{2} + 8 {D}_{g r} τ} )} {Diff}_{g r}^{GR} ( τ ) + \\ {V}_{G} K ( {c}_{g} {Diff}_{g}^{G} ( τ ) + {c}_{g r} {Diff}_{g r}^{G} ( τ ) )\end{matrix}}{\begin{matrix}( {V}_{G} ( {c}_{g} + {c}_{g r} ) + \frac{{bg}_{G}}{{W}_{ma x}^{G} {κ}_{g}^{G} {q}_{g}} ) \\ ( {V}_{R} ( {c}_{r} + {c}_{g r} ) + \frac{{bg}_{R}}{{W}_{ma x}^{R} {κ}_{r}^{R} {q}_{r}} + {V}_{G} K ( {c}_{g} + {c}_{g r} ) )\end{matrix}} \\ {G}_{R} ( τ ) - 1 = \frac{\begin{matrix}\begin{matrix}{V}_{R} ( {c}_{r} {Diff}_{r}^{R} ( τ ) + {c}_{g r} {Diff}_{g r}^{R} ( τ ) ) + \\ 2 \frac{{V}_{G} {V}_{R}}{{V}_{GR}} K {c}_{g r} {e}^{- ( \frac{2 {r}_{0}^{2}}{{ω}_{G}^{2} + {ω}_{R}^{2} + 8 {D}_{g r} τ} + \frac{2 {z}_{0}^{2}}{{z}_{G}^{2} + {z}_{R}^{2} + 8 {D}_{g r} τ} )} {Diff}_{g r}^{GR} ( τ ) +\end{matrix} \\ {V}_{G} {K}^{2} ( {c}_{g} {Diff}_{g}^{G} ( τ ) + {c}_{g r} {Diff}_{g r}^{G} ( τ ) )\end{matrix}}{{( {V}_{R} ( {c}_{g} + {c}_{g r} ) + \frac{{bg}_{R}}{{W}_{ma x}^{R} {κ}_{g}^{R} {q}_{r}} + {V}_{G} K ( {c}_{g} + {c}_{g r} ) )}^{2}} \\ {G}_{G} ( τ ) - 1 = \frac{{V}_{G} ( {c}_{g} {Diff}_{g}^{G} ( τ ) + {c}_{g r} {Diff}_{g r}^{G} ( τ ) )}{{( {V}_{R} ( {c}_{g} + {c}_{g r} ) + \frac{{bg}_{G}}{{W}_{ma x}^{G} {κ}_{g}^{G} {q}_{g}} )}^{2}}\end{matrix} \qquad ( 4 C )$$

Here, r₀=\sqrt{x₀²+y₀²} and z₀ denotes the displacement between the two lasers (in the lateral and axial dimensions, respectively) and we define the expression,

$$K = \frac{{W}_{ma x}^{G} {κ}_{g}^{R} {q}_{g}}{{W}_{ma x}^{R} {κ}_{r}^{R} {q}_{r}} or K = \frac{{W}_{ma x}^{G} {κ}_{g}^{R} {q}_{g} {σ}_{g}}{{W}_{ma x}^{R} {κ}_{r}^{R} {q}_{r} {σ}_{r}}$$

to be the crosstalk parameter, which is the ratio of the brightness of the green and red species (respectively) at the centre of each foci, when detected in the red channel. Notice that the brightness in the centre is different from the spatial average brightness (the average brightness over the whole detection volume), which we denote Q_{g}^{R}.
Further, bg_{G} and bg_{R} are the background fluorescence in the green and the red channel, respectively, when both lasers are on. Eq. 4C can easily be modified to handle the situation when diffusion is restricted to two dimensions by changing the effective volumes to areas and let z_{G} and z_{R} approach infinity.

### 4.2 FCCS Analysis

The expressions in Eq. 4C should simultaneously be fitted by a non-linear least-squares optimization routine (or similar) to the three correlation curves (G_{G}(τ), G_{R}(τ) and G_{GR}(τ)) recorded in each FCCS experiment.

### 4.3 Determination of Parameters Relevant to Quantify Interactions

The above-mentioned cross-talk and the inevitable non-perfect overlap between the two excitation laser foci are acknowledged limitations of the FCCS technique, which in general make the quantification of specific interactions difficult. In principle, the influence of cross-talk and focal displacement can be taken into account in the analysis, as stated in the refined FCCS model of Eq. 4C. However, to take advantage of this model, the displacement parameter r₀ and the cross-talk parameter K have to be known. Instead of adding complex techniques to determine the displacement or use approximate methods to determine the influence of cross-talk, it is possible with a few test measurements to find these two parameters.

### 4.3.A Determination of the Crosstalk Parameter

Cross-talk between the two fluorescent detection channels gives rise to increased apparent cross-correlation amplitude. The cross-talk parameter,

$$K = \frac{{W}_{ma x}^{G} {κ}_{g}^{R} {q}_{g}}{{W}_{ma x}^{R} {κ}_{r}^{R} {q}_{r}} , or K = \frac{{W}_{ma x}^{G} {κ}_{g}^{R} {q}_{g} {σ}_{g}}{{W}_{m ax}^{R} {κ}_{r}^{R} {q}_{r} {σ}_{r}}$$

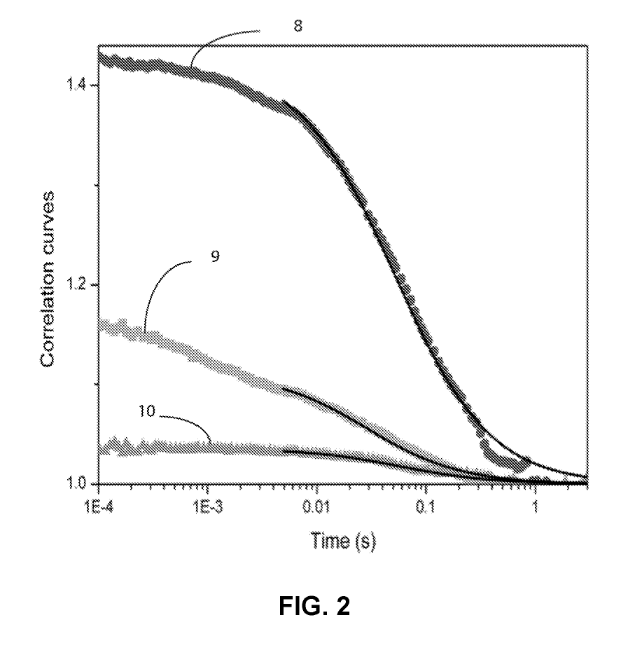

is given by the ratio between the brightness in the centre of each focus of the green (only) species and the red (only) species, as detected in the red channel for both species. It can be determined via a sample of two species lacking mutual interactions, a negative control. In this case the complex vanishes (c_{gr}=0). The rest of the parameters can be let free to vary when fitting Eq. 4C to the three correlation curves (two autocorrelation curves and one cross-correlation curve, see FIG. 2). If one is interested in the crosstalk parameter of another pair of species, then the relative brightness differences of these should be determined first (e.g. by using conventional FCS or a fluorospectrometer). The ratio of the powers of the exciting lasers is also important to adjust for if different powers are used in different experiments. With low enough excitation intensities, fluorescence saturation can be neglected. Wₘₐₓ^{G}κ_{g}^{R}q_{g} and Wₘₐₓ^{R}κᵣ^{R}qᵣ and hence also K, are then proportional to the powers of the two lasers.

To be more precise, suppose you first determine K for two species with brightness Q_{g}^{R} and Qᵣ^{R} (defined above) at a certain excitation power ratio of the lasers exciting the green and the red species and then wants to know K′ for two other fluorescently labeled molecules (which might interact with each other) with brightness Q_{g′}^{R} and Q_{r′}^{R} at possibly another power ratio. Then K′ is equal to

$${K}^{′} = K \frac{ {Q}_{{g}^{′}}^{R} / {Q}_{g}^{R}}{{Q}_{{r}^{′}}^{R} / {Q}_{r}^{R}} . \qquad ( 5 )$$

K′ could not be determined as precisely by approximating it with the expression

$$\frac{{Q}_{{g}^{′}}^{R}}{{Q}_{{r}^{′}}^{R}} .$$

When performing dual color FCCS on top of a membrane, the two laser foci are likely to be shifted slightly axially, which make such an approximation even worse.

By performing power series with different power-ratios, the accuracy in the estimation of K could be enhanced.

### 4.3.8 Determination of the Displacement Between the Focal Volumes/Areas

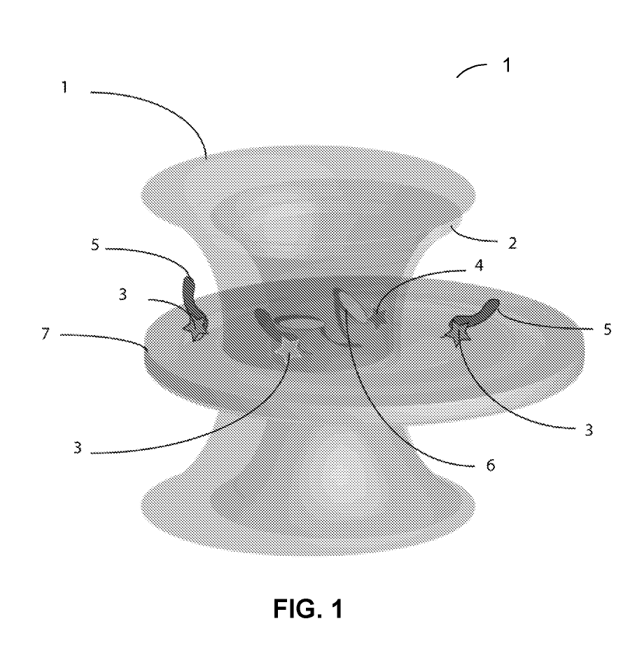

In dual laser FCCS measurements it is technically difficult to align the two lasers perfectly and a non-perfect overlap between the two detection volumes is inevitable (FIG. 1). As the distance between the laser foci increases, the amplitude of the cross-correlation will decrease. Once K is determined (see section 4.3.A), the displacement can be determined. This is achieved by performing FCCS measurements on two species that are required to interact with each other significantly. If the total concentration of the red species (cᵣ+c_{gr}) are known together with ω^{G}, ω^{R}, z_{G} and z_{R} then each concentration of the species (c_{g}, cᵣ and c_{gr}) together with their diffusion coefficients and also r₀ and z₀ could be determined by fitting the three correlation curves to Eq. 4C. The total concentration of red species (cᵣ+c_{gr}) can for example be determined by blocking the green exciting laser and use conventional FCS with the red exciting laser on the red fluorescent species.

In case of negligible K, then the displacement can be determined from the same derivations with K fixed to zero.

### 4.3.C Determination of the Focal Areas

In two dimensions it is in general difficult to calculate ω_{G} and ω_{R} on the membrane, due to the shift in the axial direction (z₀≠0) or distortions. However, if r₀ is determined from solution measurements and cᵣ vanish, then ω_{G} and ω_{R} could be estimated on the membrane or a planar surface (when we refer to a membrane, then it can equally be a surface). If cᵣ=0 and r₀, ω_{G} and ω_{R} are unknown on the membrane, then one can still get a reasonable approximation of the displacement; this is achieved by having r₀, ω_{G} and ω_{R} as free to vary parameters under the fitting procedure of the three correlation curves to Eq. 4C. The advantage is then that no solution measurements are required and the disadvantage is that ω_{G} and ω_{R} will not be correctly determined.

### 4.4 Determination of the Concentrations and the Diffusion Coefficients of the Species

With K, r₀, z₀, ω_{G}, ω_{R}, z_{G} and z_{R} determined (see section 4.3.A and 4.3.B), one can determine the concentrations and diffusion coefficients of two interacting species and their complex. This is achieved by fitting the three correlation curves to Eq. 4C and letting all unknown variables be free to vary.

### 4.5 Determination of the FRET Efficiency Upon Binding

If quenching, due to FRET, arise upon association of the labeled species, then the quantum yields multiplied with the detection efficiency of the associated complex will become a linear combination of the quantum yields multiplied with the detection efficiencies of the non-associated species. In other words; q_{gr}κ_{gr}^{G} and q_{gr}κ_{gr}^{R} will become linear combinations of q_{g}κ_{g}^{G} and qᵣκᵣ^{R}. These linear combinations can then substitute q_{gr}κ_{gr}^{G} and q_{gr}κ_{gr}^{R} in Eq. 4C.

If the parameters K, r₀, z₀, ω^{G}, ω_{R}, z_{G} and z_{R} are determined beforehand (see previous sections) then, if FRET is present, parameters related to the FRET efficiency can be determined by fitting Eq. 4, with the appropriate substitutions of q_{gr}κ_{gr}^{G} and q_{gr}κ_{gr}^{R}, to the three correlation curves keeping the known parameters fixed.

### EXAMPLES

The following non-limiting examples will further illustrate the present invention.

### 4.6 Example

### Determination of the Displacement Using Fluorescently Labelled Complementary DNA Strands

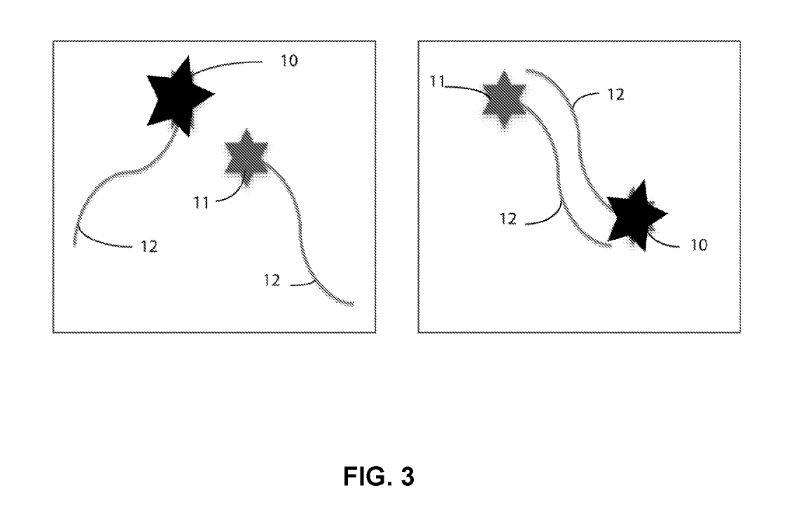

See FIG. 3. Two short complementary DNA strands can efficiently determine the displacement in the lateral as well as in the axial direction in a dual color FCCS setup.
1) Label one type of strand with a green fluorescent dye and the other with a red fluorescent dye.
2) Denature (break the hydrogen bonds) the two strands and measure on the mixed strands with FCCS. Fit the data according to section 4.2, to retrieve the cross-talk parameter K.
3) Take a sample, where the two strands are bound to each other. Block the laser exciting the green emitting dye and determine the total concentration of red species (which includes both r and gr species) with FCS. Then unblock the green laser and record the FCCS curves. Fit the data according to section 4.2 with K fixed to the value determined from step 2.

From these steps the lateral (r₀) as well as the axial (z₀) displacement is found. It is tacitly understood that the labeling should be such that no FRET occurs as the two strands are bound to each other.

### 4.7.A.a Example

### Determination of the Focal Areas on a Membrane Using Fluorescently Labeled Antibodies or Other Affinity Molecules

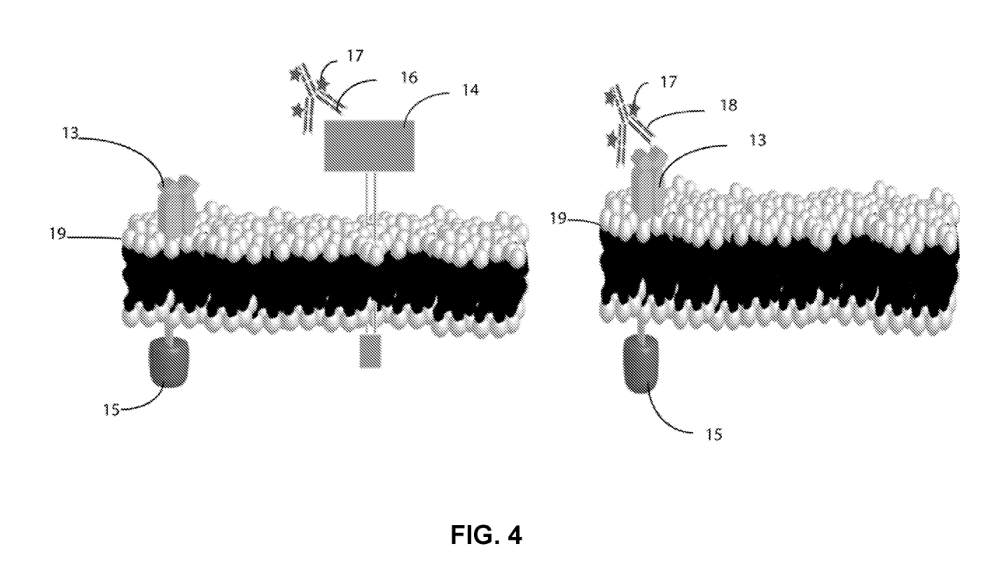

Suppose that two particular membrane proteins, A and B, within a cell line, are known not to interact with each other (see FIG. 4). Assume that the A-protein is fused with a green tag and that antibodies, Fab fragments, or other binding reagents with high specificity and affinity, exist for both proteins. The binding reagents are called affinity molecules below. The displacement can be found beforehand by using Ex. 4.6. Performing the following steps will determine the focal width parameters w_{G} and w_{R}:
1) Label both affinity molecules with the same type of dye and determine the brightness ratio between them (by using, for example, FCS).
2) Add the labeled B-affinity molecule to the cell sample (lacking A-affinity molecules) and measure on the membrane of the cell with FCCS (see FIG. 4, Left). Fit the data according to section 4.2 and extract the cross-talk parameter K.
3) Add the labeled A-affinity molecule to the cell sample (lacking B-affinity molecules) and measure with FCCS (see FIG. 4, Right). Fit the data according to section 4.2 with K fixed to the value determined from step 2 corrected with the brightness ratio found in step 1 (use Eq. 5 for brightness correction).

The output from step 3 is the parameters ω_{G} and ω_{R}.

One can actually get reasonable value for the lateral displacement if it is let free to vary in the fitting procedure. This was actually taken into advantage of in the application of section 4.8.

### 4.7.A.b Example

### Determination of the Focal Areas on a Membrane Using Proteins which are Intrinsically Labeled with a Fluorescent Tag

Suppose that two particular membrane proteins, C and D, within a cell line, are known not to interact with each other. The C-protein is by genetic engineering produced in two variants, one fused with a green label, and one variant fused with both a green and a red label. Protein D is produced coupled to a red label. The displacement can be found beforehand by using Ex. 4.6. Performing the following steps will determine the focal width parameters w_{G} and w_{R}:
4) Determine the brightness ratio between the fluorescent label (by using, for example, FCS).
5) Perform FCCS measurements on the membrane of cells expressing protein C coupled only to the green label, and protein D coupled to the red label. Fit the data according to section 4.2 and extract the cross-talk parameter K.
6) Perform FCCS measurements on the same type of cells, but expressing only protein C, coupled to the green and red label. Fit the data according to section 4.2 with K fixed to the value determined from step 2 corrected with the brightness ratio found in step 1 (use Eq. 5 for brightness correction).

Another alternative is to produce intrinsically labeled variants of affinity molecules, one green labeled and one green-and-red labeled affinity molecule towards protein C, and one red labeled affinity molecule against protein D, and then follow the steps above with a cell line expressing both proteins, but instead alternate the addition of affinity molecules. Important in both variants is that each and every of the double-labelled molecules carries at least one green label.

### 4.8 Example

### Determination of the FRET Efficiency Between to Fluorescently Labeled Proteins

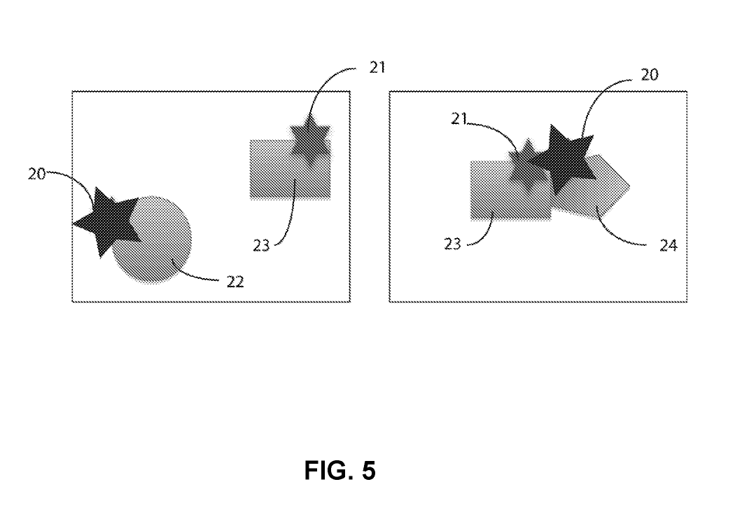

Suppose a green fluorescent labeled protein A is interacting with a red fluorescent labeled protein B (see FIG. 5). Assume that there exists another protein C, also labeled with a red fluorescent label, which is known not to interact with protein A. Assume that r₀, z₀, ω_{G}, ω_{R}, z_{G} and z_{R} are known (conventional FCS measurements for determine all parameters except r₀ and z₀, which can be derived according to Example 4.6). To be able to determine the FRET efficiency of the AB interaction, then follow:
1) Determine the relative brightness difference between the labeled B and C proteins;
2) Measure with FCCS on a sample containing both the A and C molecules. Fit the curves according to section 4.2 and extract the crosstalk parameter and modify it according to Eq. 5
3) Measure with FCCS on a mixture of A and B molecules. Fit the curves, with K as determined from step 2, according to section 4.2 with Eq. 4c in combination with the corrections (according to section 4.5).

The output from step 3 are parameters related to FRET (see section 4.5).

### 4.9. Detailed Description on the Derivation of the Dual Color Cross-Correlation Expression for a Nonperfect Overlap, and when Brightness Differences and Cross Talk are Present

The detected fluorescence fluctuations, ∂F_{G}(t) and ∂F_{R}(t) from a set of green species and a set of red species are given by:

$$\begin{matrix} \partial {F}_{G} ( t ) = {\sum }_{u ∈ {Set}_{G}} {k}_{u}^{G} {\int }_{{R}^{3}} {W}_{G} ( \overline{r} ) \partial {c}_{u} ( \overline{r} , t ) {\partial }^{3} r & ( A 1 ) \\ \partial {F}_{R} ( t ) = {\sum }_{u ∈ {Set}_{R}} {k}_{u}^{R} {\int }_{{R}^{3}} {W}_{R} ( \overline{r} - {\overline{r}}_{0} ) \partial {c}_{u} ( \overline{r} , t ) {\partial }^{3} r + {\sum }_{u ∈ {Set}_{G}} {u}_{R}^{} {\int }_{{R}^{3}} {W}_{G} ( \overline{r} ) \partial {c}_{u} ( \overline{r} , t ) {\partial }^{3} r . & ( A 2 )\end{matrix}$$

In Eq. A1 and A2 the following abbreviations have been used:

i) Set_{G} and Set_{R} are the sets of the green and red species, respectively. In case of three species, g, r and gr, then Set_{G}={g, gr} and Set_{R}={r, gr}.
ii) k=κ·q, is the product of the detection efficiency (κ) and the fluorescence quantum yield (q). More precisely: kᵤ^{G} is the probability of detecting a fluorescence photon in the green detector when a photon has been absorbed by the green fluorophore of the species u. kᵤ^{R} is the probability of detecting a fluorescence photon in the red detector when a photon has been absorbed by the red fluorophore of the species u. kᵤ^{G} is the probability of detecting a fluorescence photon in the red detector when a photon has been absorbed by the green fluorophore (due to crosstalk) of the species u.
iii) ∂cᵤ is the concentration fluctuations of species u.
iv) W_{G}( r̅)=CEF_{G} ( r̅)I_{exc,G}( r̅) and W_{R}( r̅)=CEF_{R} ( r̅)I_{exc,R}( r̅) are the green and the red fluorescence brightness distributions, respectively. Where I_{exc,G}( r̅) and I_{exc,R}( r̅) denote the excitation intensity of the laser exciting the green and the red species, respectively, and CEF_{G}( r̅) and CEF_{R}( r̅) signifies the collection efficiency function of the instrument in each colour range;
v) r̅₀ is the displacement parameter, which is the distance between the centre of the green and the red laser focus.

In Eq. A2, the second summation term is due to cross-talk from the green species, detected in the red channel. The normalized CEF_{R}( r̅)I_{exc,G}( r̅) is assumed to be equal to the normalized CEF_{G}( r̅)I_{exc,G}( r̅).

Inserting Eq. A1 and Eq. A2 into the cross-correlation of the fluorescence (Eq. 3) yields:

$${G}_{GR} ( τ ) - 1 = \frac{〈 \begin{matrix}( {\sum }_{u ∈ {Set}_{G}} {k}_{u}^{G} {\int }_{{R}^{3}} {W}_{G} ( \overline{r} ) \partial {c}_{u} ( \overline{r} , t + τ ) {\partial }^{3} r ) \\ ( \begin{matrix}{\sum }_{u ∈ {Set}_{R}} {k}_{u}^{R} {\int }_{{R}^{3}} {W}_{R} ( \overline{r} - {\overline{r}}_{0} ) \partial {c}_{u} ( \overline{r} , t ) {\partial }^{3} r + \\ {\sum }_{u ∈ {Set}_{G}} {u}_{R}^{} {W}_{G} ( \overline{r} ) \partial {c}_{u} ( \overline{r} , t ) {\partial }^{3} r\end{matrix} )\end{matrix} 〉}{\begin{matrix}( {\sum }_{u ∈ {Set}_{G}} {k}_{u}^{G} {\int }_{{R}^{3}} {W}_{G} ( \overline{r} ) {\partial }^{3} r ) \\ ( {\sum }_{u ∈ {Set}_{R}} {k}_{u}^{R} {c}_{u} {\int }_{{R}^{3}} {W}_{R} ( \overline{r} ) {\partial }^{3} r + {\sum }_{u ∈ {Set}_{G}} {u}_{R}^{} {c}_{u} {\int }_{{R}^{3}} {W}_{G} ( \overline{r} ) {\partial }^{3} r )\end{matrix}} \qquad ( A3 )$$

The concentration fluctuations of two different species u and v are always uncorrelated if they are not interacting with each other, i.e. <∂cᵤ( r̅′,t+τ)∂cᵥ( r̅,t)>=0 Hence:

$${G}_{GR} ( τ ) - 1 = \frac{( \begin{matrix}\begin{matrix}{\sum }_{u ∈ {Set}_{G} ⋂ {Set}_{R}} {k}_{u}^{G} {k}_{u}^{R} {\int }_{{R}^{3}} {\int }_{{R}^{3}} {W}_{G} ( \overline{r} ) {W}_{R} ( {\overline{r}}^{′} - {\overline{r}}_{0} ) \\ 〈 \partial {c}_{u} ( \overline{r} , t + τ ) \partial {c}_{u} ( {\overline{r}}^{′} , t ) 〉 {\partial }^{3} r {\partial }^{3} {r}^{′} +\end{matrix} \\ \begin{matrix}{\sum }_{u ∈ {Set}_{G}} {k}_{u}^{G} {u}_{R}^{} {\int }_{{R}^{3}} {\int }_{{R}^{3}} {W}_{G} ( \overline{r} ) {W}_{G} ( {\overline{r}}^{′} ) \\ 〈 \partial {c}_{u} ( \overline{r} , t + τ ) , \partial {c}_{u} ( {\overline{r}}^{′} , t ) 〉 {\partial }^{3} r {\partial }^{3} {r}^{′}\end{matrix}\end{matrix} )}{\begin{matrix}( {\sum }_{u ∈ {Set}_{G}} {k}_{u}^{G} {c}_{u} {\int }_{{R}^{3}} {W}_{G} ( \overline{r} ) {\partial }^{3} r ) \\ ( \begin{matrix}{\sum }_{u ∈ {Set}_{R}} {k}_{u}^{R} {c}_{u} {\int }_{{R}^{3}} {W}_{R} ( \overline{r} ) {\partial }^{3} r + \\ {\sum }_{u ∈ {Set}_{G}} {u}_{R}^{} {c}_{u} {\int }_{{R}^{3}} {W}_{G} ( \overline{r} ) {\partial }^{3} r\end{matrix} )\end{matrix}} \qquad ( A4 )$$

Here, Set_{G}∩Set_{R}, is not empty if there are double labelled species. According to Parsevals theorem:

$${\int }_{- ∞}^{∞} f ( x ) {g ( x )}^{*} d x = {\int }_{- ∞}^{∞} {F}_{v} [ f ( x ) ] {{F}_{v} [ g ( x ) ]}^{*} d v , \\ {F}_{v} [ f ( x ) ] = {\int }_{- ∞}^{∞} f ( x ) {e}^{- j vx} d x$$

denotes the Fourier transform of f(x), and star (*) indicates complex conjugation. Hence:

$${G}_{GR} ( τ ) - 1 = \frac{( \begin{matrix}{\sum }_{u ∈ {Set}_{G} ⋂ {Set}_{R}} {k}_{u}^{G} {k}_{u}^{R} {\int }_{{R}^{3}} {\int }_{{R}^{3}} {W}_{G} ( \overline{r} ) {F}_{v} [ {W}_{R} ( {\overline{r}}^{′} - {\overline{r}}_{0} ) ] {{F}_{v} [ 〈 \partial {c}_{u} ( \overline{r} , t + τ ) \partial {c}_{u} ( {\overline{r}}^{′} , t ) 〉 ]}^{*} {\partial }^{3} r {\partial }^{3} v + \\ {\sum }_{u ∈ {Set}_{G}} {k}_{u}^{G} {u}_{R}^{} {\int }_{{R}^{3}} {\int }_{{R}^{3}} {W}_{G} ( \overline{r} ) {F}_{v} [ {W}_{G} ( {\overline{r}}^{′} ) ] {{F}_{v} [ 〈 \partial {c}_{u} ( \overline{r} , t + τ ) , \partial {c}_{u} ( {\overline{r}}^{′} , t ) 〉 ]}^{*} {\partial }^{3} r {\partial }^{3} v\end{matrix} )}{( \begin{matrix}( {\sum }_{u ∈ {Set}_{G}} {k}_{u}^{G} {c}_{u} {\int }_{{R}^{3}} {W}_{G} ( \overline{r} ) {\partial }^{3} r ) \\ ( {\sum }_{u ∈ {Set}_{R}} {k}_{u}^{R} {c}_{u} {\int }_{{R}^{3}} {W}_{R} ( \overline{r} ) {\partial }^{3} r + {\sum }_{u ∈ {Set}_{G}} {u}_{R}^{} {c}_{u} {\int }_{{R}^{3}} {W}_{G} ( \overline{r} ) {\partial }^{3} r )\end{matrix} )} = \frac{( \begin{matrix}{\sum }_{u ∈ {Set}_{G} ⋂ {Set}_{R}} {k}_{u}^{G} {k}_{u}^{R} {\int }_{{R}^{3}} {\int }_{{R}^{3}} {W}_{G} ( \overline{r} ) {e}^{- j {\overline{vr}}_{0}} {F}_{v} [ {W}_{R} ( {\overline{r}}^{′} ) ] {c}_{u} {e}^{- {D}_{u} {\overline{v}}^{2} τ} {e}^{- j \overline{vr}} {\partial }^{3} r {\partial }^{3} v + \\ {\sum }_{u ∈ {Set}_{G}} {k}_{u}^{G} {u}_{R}^{} {\int }_{{R}^{3}} {\int }_{{R}^{3}} {W}_{G} ( \overline{r} ) {F}_{v} [ {W}_{G} ( {\overline{r}}^{′} ) ] {c}_{u} {e}^{- {D}_{u} {\overline{v}}^{2} τ} {e}^{- j \overline{vr}} {\partial }^{3} r {\partial }^{3} v\end{matrix} )}{( \begin{matrix}( {\sum }_{u ∈ {Set}_{G}} {k}_{u}^{G} {c}_{u} {\int }_{{R}^{3}} {W}_{G} ( \overline{r} ) {\partial }^{3} r ) \\ ( {\sum }_{u ∈ {Set}_{R}} {k}_{u}^{R} {c}_{u} {\int }_{{R}^{3}} {W}_{R} ( \overline{r} ) {\partial }^{3} r + {\sum }_{u ∈ {Set}_{G}} {u}_{R}^{} {c}_{u} {\int }_{{R}^{3}} {W}_{G} ( \overline{r} ) {\partial }^{3} r )\end{matrix} )} = \frac{( \begin{matrix}{\sum }_{u ∈ {Set}_{G} ⋂ {Set}_{R}} {k}_{u}^{G} {k}_{u}^{R} {\int }_{{R}^{3}} {F}_{v} [ {W}_{G} ( \overline{r} ) ] {F}_{v} [ {W}_{G} ( {\overline{r}}^{′} ) ] {c}_{u} {e}^{- {D}_{u} {\overline{v}}^{2} τ - j {\overline{vr}}_{0}} {\partial }^{3} v + \\ {\sum }_{u ∈ {Set}_{G}} {k}_{u}^{G} {u}_{R}^{} {\int }_{{R}^{3}} {F}_{v} [ {W}_{G} ( \overline{r} ) ] {F}_{v} [ {W}_{G} ( {\overline{r}}^{′} ) ] {c}_{u} {e}^{- {D}_{u} {\overline{v}}^{2} τ} {\partial }^{3} v\end{matrix} )}{( \begin{matrix}( {\sum }_{u ∈ {Set}_{G}} {k}_{u}^{G} {c}_{u} {\int }_{{R}^{3}} {W}_{G} ( \overline{r} ) {\partial }^{3} r ) \\ ( {\sum }_{u ∈ {Set}_{R}} {k}_{u}^{R} {c}_{u} {\int }_{{R}^{3}} {W}_{R} ( \overline{r} ) {\partial }^{3} r + {\sum }_{u ∈ {Set}_{G}} {u}_{R}^{} {c}_{u} {\int }_{{R}^{3}} {W}_{G} ( \overline{r} ) {\partial }^{3} r )\end{matrix} )} \qquad ( A6 )$$

In Eq. A6, standard rules for Fourier transforms were applied, except for

Fᵥ[ [symbol] ∂cᵤ( r̅′,t)∂cᵤ( r̅,t+τ) [symbol] ]=cᵤe^{-D}^{ᵤ}^{\vec{v}}^{²}^{τ}e^{-j v̅r̅}. (A7)

which is derived in reference [6].
Now, suppose that the excitation profile has a gaussian distribution, i.e.

$$W ( x , y , z ) = {W}_{max} {e}^{- 2 ( \frac{{x}^{2} + {y}^{2}}{{ω}_{xy}^{2}} + \frac{{z}^{2}}{{ω}_{z}^{2}} )} .$$

Then Fᵥ[W( r̅)]=Wₘₐₓω²z₀e^{-(v}^{ₓ}^{²}^{ω}^{_{xy}}^{²}^{+v}^{_{y}}^{²}^{ω}^{_{xy}}^{²}^{+v}^{_{z}}^{²}^{ω}^{_{z}}^{²}^{)/8}/8,

$${\int }_{{R}^{3}} W ( \overline{r} ) {\partial }^{3} r = {{W}_{max} ( \frac{π}{2} )}^{\frac{3}{2}} {ω}_{xy}^{2} {ω}_{z}$$

and Eq. A6 can be transformed into:

$${G}_{GR} ( τ ) - 1 = \frac{( \begin{matrix}{\sum }_{u ∈ {Set}_{G} ⋂ {Set}_{R}} {k}_{u}^{G} {k}_{u}^{R} {\int }_{{R}^{3}} \frac{{ω}_{G}^{2} {z}_{G} {ω}_{R}^{2} {z}_{R} {c}_{u} {e}^{- ( \begin{matrix}{v}_{x}^{2} ( {ω}_{G}^{2} + {ω}_{R}^{2} ) / 8 + {v}_{y}^{2} ( {ω}_{G}^{2} + {ω}_{R}^{2} ) / 8 + \\ {v}_{z}^{2} ( {z}_{G}^{2} + {z}_{R}^{2} ) / 8 + {D}_{u} {\overline{v}}^{2} τ + j {\overline{vr}}_{0}\end{matrix} )}}{{8}^{2}} {\partial }^{3} v + \\ {\sum }_{u ∈ {Set}_{G}} {k}_{u}^{G} {{k}_{u}^{R} ( \frac{{W}_{max}^{G}}{{W}_{max}^{R}} )}^{2} {\int }_{{R}^{3}} {\int }_{{R}^{3}} \frac{{ω}_{G}^{4} {z}_{G}^{2} {c}_{u} {e}^{- ( \begin{matrix}{v}_{x}^{2} {ω}_{G}^{2} / 4 + 2 {v}_{y}^{2} {ω}_{G}^{2} / 4 + \\ 2 {v}_{z}^{2} {z}_{G}^{2} / 4 + {D}_{u} {\overline{v}}^{2} τ\end{matrix} )}}{{8}^{2}} {\partial }^{3} v\end{matrix} )}{( {( \frac{π}{2} )}^{3 / 2} {ω}_{G}^{2} {z}_{G} {\sum }_{u ∈ {Set}_{G}} {k}_{u}^{G} {c}_{u} ) ( {( \frac{π}{2} )}^{3 / 2} {ω}_{R}^{2} {z}_{R} {\sum }_{u ∈ {Set}_{R}} {k}_{u}^{R} {c}_{u} + {( \frac{π}{2} )}^{3 / 2} {ω}_{G}^{2} {z}_{G} \frac{{W}_{max}^{G}}{{W}_{max}^{R}} {\sum }_{u ∈ {Set}_{G}} 𝒦 {/}_{u}^{R} {c}_{u} )} \qquad ( A 8 )$$

By integrating along a rectangular contour in the complex plane, the complex integral of Eq. A8 can be written:

$${\int }_{- ∞}^{∞} {e}^{- ( {v}_{x}^{2} ( {ω}_{G}^{2} + {ω}_{R}^{2} ) / 8 + {D}_{u} {v}_{x}^{2} τ + j {v}_{x} {x}_{0} )} \partial {v}_{x} = \sqrt{\frac{8 π}{{ω}_{G}^{2} + {ω}_{R}^{2}}} {{e}^{- \frac{2 {x}_{0}^{2}}{{ω}_{G}^{2} + {ω}_{R}^{2} + 8 {D}_{u}}} ( 1 + \frac{8 {D}_{u} τ}{{ω}_{G}^{2} + {ω}_{R}^{2}} )}^{- \frac{1}{2}} . \qquad ( A9 )$$

Inserting Eq. A9 into Eq. A8 yields the following expression for the cross correlation:

$$\begin{matrix}{G}_{GR} ( τ ) - 1 = \frac{\begin{matrix}( \frac{{V}_{G} {V}_{R}}{{V}_{GR}} {\sum }_{u ∈ {Set}_{G} ⋂ {Set}_{R}} {k}_{u}^{G} {k}_{u}^{R} {c}_{u} {e}^{- \frac{2 {x}_{0}^{2} + 2 {y}_{0}^{2}}{{ω}_{G}^{2} {ω}_{R}^{2} + 8 {D}_{u} τ} - \frac{2 {z}_{0}^{2}}{{z}_{G}^{2} + {z}_{R}^{2} + 8 {D}_{u} τ}} {Diff}_{u}^{GR} ( τ ) ) + \\ ( {V}_{G} \frac{{W}_{max}^{G}}{{W}_{max}^{R}} {\sum }_{u ∈ {Set}_{G}} {k}_{u}^{G} k {/}_{u}^{R} {c}_{u} {Diff}_{u}^{G} ( τ ) )\end{matrix}}{( ( {V}_{G} {\sum }_{u ∈ {Set}_{G}} {k}_{u}^{G} {c}_{u} ) ( {V}_{R} {\sum }_{u ∈ {Set}_{R}} {k}_{u}^{R} {c}_{u} + {V}_{G} \frac{{W}_{max}^{G}}{{W}_{max}^{R}} {\sum }_{u ∈ {Set}_{G}} k {/}_{u}^{R} {c}_{u} ) )} & ( A10 ) \\ {Diff}_{u}^{G} = {( 1 + \frac{4 {D}_{u} τ}{{ω}_{G}^{2}} )}^{- 1} {( 1 + \frac{4 {D}_{u} τ}{{z}_{G}^{2}} )}^{- 1 / 2} \\ {Diff}_{u}^{R} = {( 1 + \frac{4 {D}_{u} τ}{{ω}_{R}^{2}} )}^{- 1} {( 1 + \frac{4 {D}_{u} τ}{{z}_{R}^{2}} )}^{- 1 / 2} \\ {Diff}_{u}^{GR} = {( 1 + \frac{8 {D}_{u} τ}{{ω}_{G}^{2} + {ω}_{R}^{2}} )}^{- 1} {( 1 + \frac{8 {D}_{u} τ}{{z}_{G}^{2} + {z}_{R}^{2}} )}^{- 1 / 2} & ( A10B ) \\ {V}_{G} = {π}^{3 / 2} {ω}_{G}^{2} {z}_{G} \\ {V}_{R} = {π}^{3 / 2} {ω}_{R}^{2} {z}_{R} \\ {V}_{GR} = {( \frac{π}{2} )}^{3 / 2} ( {ω}_{G}^{2} + {ω}_{R}^{2} ) \sqrt{{z}_{G}^{2} + {z}_{R}^{2}} & ( A10C )\end{matrix}$$

With the same approach, as just been described, the autocorrelation functions of the green and red fluorescence were obtained:

$$\begin{matrix}{G}_{R} ( τ ) - 1 = \frac{\begin{matrix}( {V}_{R} {\sum }_{u ∈ {Set}_{R}} {( {k}_{u}^{R} )}^{2} {c}_{u} {Diff}_{u}^{R} ( τ ) ) + \\ ( 2 \frac{{V}_{G} {V}_{R}}{{V}_{GR}} \frac{{W}_{max}^{G}}{{W}_{max}^{R}} {\sum }_{u ∈ {Set}_{G} ⋂ {Set}_{R}} k {/}_{u}^{R} {k}_{u}^{R} {c}_{u} {Diff}_{u}^{GR} ( τ ) {e}^{- \frac{2 ( {x}_{0}^{2} + 2 {y}_{0}^{2} )}{{ω}_{G}^{2} {ω}_{R}^{2} + 8 {D}_{u} τ} - \frac{2 {z}_{0}^{2}}{{z}_{G}^{2} + {z}_{R}^{2} + 8 {D}_{u} τ}} ) + \\ ( {{V}_{G} ( \frac{{W}_{max}^{G}}{{W}_{max}^{R}} )}^{2} {\sum }_{u ∈ {Set}_{G}} {( k {/}_{u}^{R} )}^{2} {c}_{u} {Diff}_{u}^{GR} ( τ ) )\end{matrix}}{{( {V}_{R} {\sum }_{u ∈ {Set}_{R}} {k}_{u}^{R} {c}_{u} + {V}_{G} \frac{{W}_{max}^{G}}{{W}_{max}^{R}} {\sum }_{u ∈ {Set}_{G}} k {/}_{u}^{R} {c}_{u} )}^{2}} & ( A11 ) \\ {G}_{G} ( τ ) - 1 = \frac{{V}_{G} {\sum }_{u ∈ G} ( {k}_{u}^{G} ) {c}_{u} {Diff}_{u}^{G} ( τ )}{{( {V}_{G} {\sum }_{u ∈ G} {k}_{u}^{G} {c}_{u} )}^{2}} & ( A12 )\end{matrix}$$

Note that the background intensities are implicitly included in the general correlation expressions of Eq. A10A, A11 and A12, since background intensities can be regarded as species with infinite diffusion coefficients.

In the case of two-dimensional diffusion, the effective volumes (V_{G}, V_{R} and V_{GR}) should be replaced with the effective areas (A_{G}, A_{R} and A_{GR}) in Eq. A10A, A11 and A12, and z_{G} and z_{R} should be replaced with infinity in Eq. A10A, A10B, A11, and A12.

### 5. Experimental Example

### Determination of Concentrations and Diffusion Coefficients of Two Interacting Species in the Membrane of Live Cells

### Materials and Methods

### Cell Lines

The murine lymphoma cell line EL-4 was used for all measurements. This cell line spontaneously expresses the MHC class I molecules H-2K^{b} and H-2D^{b}, but not H-2D^{d}. Due to its presumed origin as a natural killer T (NKT) lymphoma, the Ly49A receptor is spontaneously expressed in a variable way on these cells. The EL4 cell line was previously transfected with a fusion protein between H-2D^{d} and EGFP (from hereon called D^{d}-EGFP). Cells were grown in RPMI medium supplemented with 10% fetal calf serum, 2 mM L-glutamine, 100 U/ml penicillin, 100 μg/ml streptomycin, 100 μM non-essential amino acids, and 1 mM sodium pyruvate (Invitrogen).

### Antibodies and Staining Procedure

Anti-H-2D^{d} (clone 34-2-12), anti-H-2K^{b} (clone AF6-88.5), and anti-Ly49A (clone JR9-318) monoclonal antibodies were purchased from BD Biosciences/Pharmingen. The JR9-318 antibody binds Ly49A regardless of whether it is free or associated with H-2D^{d} in cis, in contrast to other available Ly49A antibodies. All antibodies were labelled with Alexa-647 using an “Alexa 647 Monoclonal Antibody Labelling Kit” (Invitrogen), following the manufacturer's protocol. The labelled antibodies were analyzed for labelling efficiency measuring absorbance in a Microdrop spectrophotometer (Microdrop Technologies GmbH, Germany) and by FCS, yielding equivalent results for the labelling efficiency. On average, the anti-Ly49A antibody (Ly49A-ab) was found to have ˜4 fluorophores per antibody, the anti-K^{b} antibody (K^{b}-ab) ˜1 and the anti-D^{d} antibody (D^{d}-ab), labelled in two different batches, ˜3 and ˜6 fluorophores per antibody, respectively. The brightness ratios (which were later on used for calculating the cross-talk parameter) were 3.8 for Ly49A and either 3.3 or 6.4 for the two D^{d}-ab:s, compared to the K^{b}-ab. For the cellular measurements, cells were stained with around 10 μg/ml antibody in phosphate buffered saline (PBS) and washed by centrifugation.

### Characterization of the Expression Patterns of the Involved Molecules in the EL4 Cell Line.

For flow cytometry, a FACS Calibur was used (BD Biosciences). D^{d}-EGFP fluorescence was detected on the majority of the EL4 cells in the culture, but the expression level varied over a large range between the cells. The origin of this heterogeneity is unknown, but it was stable over time in the cell culture. The EGFP fluorescence in each cell was proportional to the fluorescence using an Al647-conjugated antibody against H-2D^{d}, suggesting expressed D^{d}-EGFP-molecules were well localized to the cell surface and the majority of H-2D^{d} molecules had a functional EGFP entity. The density of Ly49A showed a similar intra-cellular variation. The expression density of Ly49A was found to be independent of the H-2D^{d} expression level. Cells expressing different combinations of Ly49A and H-2D^{d} at various densities could thus easily be found within the same cell line. This variability was taken advantage of to quantify the cis interaction between H-2D^{d} and Ly49A. H-2K^{b} was expressed at a more homogeneous concentration at the cell population level. However, there was enough spread in intra-cellular H-2K^{b} concentration to allow a range of measurements at different concentrations, matching the concentration ranges of H-2D^{d} and Ly49A.

### FCS Equipment and Settings

Fluorescence microscopy and FCS measurements were performed on a Confocor 3 system (Zeiss, Jena, Germany). An Ar-Ion laser (488 nm) and a HeNe laser (633 nm) were focused through a C-Apochromat 40×, NA 1.2 objective. The fluorescence was detected by two avalanche photodiodes after passage through a dichroic mirror (HFT 488/543/633), a pinhole (edge-to-edge distance 70 μm for the FCS and 300 μm for the fluorescence microscope) in the image plane, a beam splitter (NFT 545) and an emission filter in front of each detector (BP 505-530 IR and LP655). The excitation power before the objective was within the range of 1 to 10 μW for the 488 nm-line and 0.5 to 3.5 μW for the 633 nm-line.

EL-4 cells were stained with antibodies as described above. Consecutively, 50000 cells per chamber were suspended in PBS and distributed in Lab-Tek 8-well chamber-glasses (Nunc, Thermo Scientific, Langenselbold, Germany). Data was acquired in 10 second intervals. Collection intervals containing abnormal fluorescence peaks, presumably resulting from aggregates, or within which the overall fluorescence was decaying, putatively due to membrane movements, were discarded from the overall analysis. The total included measurement times ranged from 50 to 100 s per cell. Measurements were only undertaken on viable cells where the D^{d}-EGFP was well localized to the cell surface membrane, as judged by visual inspection in the wide-field and confocal mode. The autofluorescence, as well as the fluorescence from both EGFP and Alexa647 in the extracellular liquid and the intracellular region was negligible (data not shown).

### FCS Analysis

The expressions in Eq. 4C were simultaneously fitted by a non-linear least-squares optimization routine (Origin 8, OriginLab Corporation, Northampton, Mass., USA) to the three correlation curves (G_{G}(τ), G_{R}(τ) and G_{GR}(τ)) recorded in each FCCS experiment. In the analysis, a three-step procedure was followed where a set of experimental parameters was first determined by negative and positive control experiments, before the actual cis-interaction was assessed:

1) In the negative control FCCS experiments (with no gr species)ω_{G} and ω_{R} were fixed to the corresponding values found in the solution measurements of that particular measurement day, and c_{gr} was fixed to zero. The rest of the parameters were free to vary.
2) In the positive control experiments (with gr but with no r species), K was fixed to the brightness corrected average value determined from the negative controls and cᵣ was fixed to zero. The rest of the variables were free to vary.
3) In the cis-interaction measurements, K was fixed to the brightness corrected average value determined from the negative controls, and the parameters r₀, ω_{G} and ω_{R} were all fixed to the average values determined from the positive controls of that particular measurement day. The rest of the variables were free to vary.

In the positive control, the signal from D^{d}-EGFP was detected in combination with an antibody against the very same H-2D^{d} molecule. As a negative control, the signal from D^{d}-EGFP was combined with detection of an antibody against H-2K^{b}, which does not interact with H-2D^{d}. For the detection and quantification of cis-interaction between D^{d}-EGFP and Ly49A, the signal from D^{d}-EGFP was combined with an antibody against Ly49A.

All measurements displayed an excitation dependent dark state of EGFP in the green auto-correlation curves (G_{G}(τ)) with a relaxation time in the ˜0.5 ms range, as previously observed. To avoid any influence from this process, the fitting of the parameters in Eq. 4C to the experimental correlation curves was restricted to correlation times longer than 5 ms. In all fittings, bg_{G} and bg_{R} were fixed to zero, due to the negligible background fluorescence.

### Results and Discussion

### Strategy

The aim of this study was to detect and quantify the amount of interaction between the NK cell receptor Ly49A, and its ligand, the MHC class I allele H-2D^{d}, within the plasma membrane of a single cell (so-called cis interaction). By labelling the two interaction partners and using dual colour FCCS, not only the fraction of cis-associated Ly49A could be determined, but also the concentrations and diffusion coefficients of all three species (Ly49A, H-2D^{d} and their cis-associated complex).

### Detection of Cis Interaction Between Ly49A and H-2D^{d}

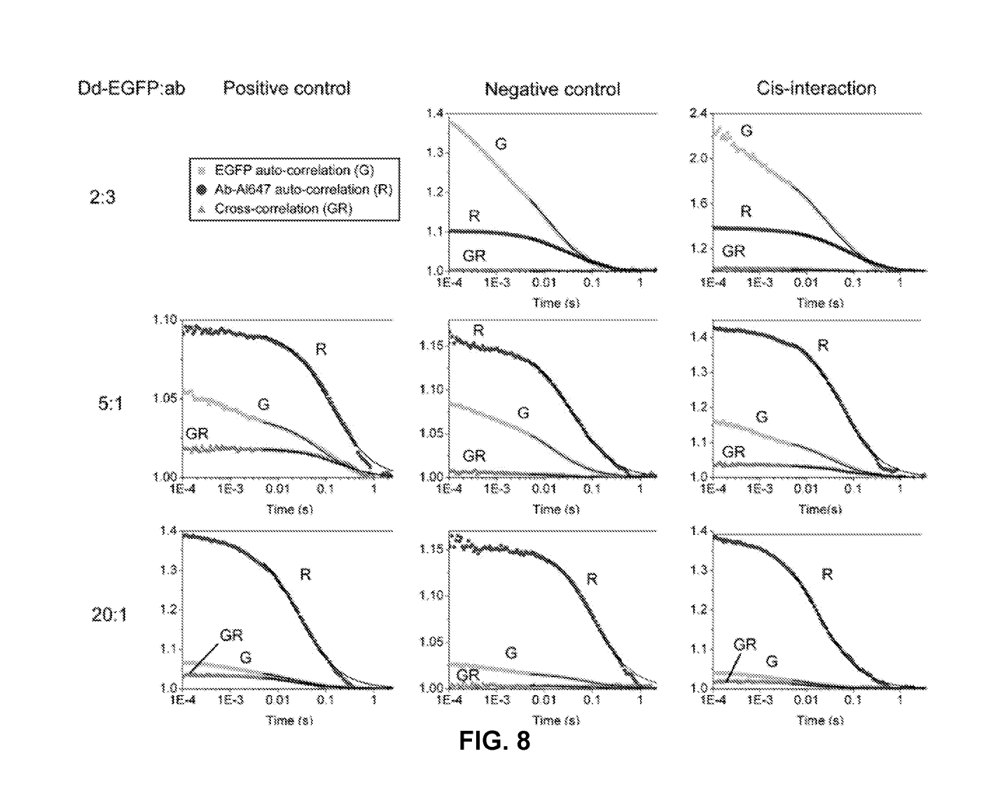

Following the strategy above, FCCS measurements were performed with fluorescence fluctuations from D^{d}-EGFP correlated with those from Al647-ab:s directed against either H-2D^{d}, H-2K^{b}, or Ly49A. Data was collected from a number of cells for each combination, displaying different concentrations of D^{d}-EGFP and Al647-ab. In FIG. 8, representative auto- and cross-correlations curves for test samples and controls are shown. Each row represents a certain concentration ratio between D^{d}-EGFP (ligands) and the respective Al647-antibody. In this way, the amount of cross-correlation can be compared between controls and test samples under equivalent concentration ratios.

In the top row, typical cells having 1-2 times more antibodies than D^{d}-EGFP are shown. This situation did not exist for the positive control, since the D^{d}-ab:s were limited by the number of D^{d}-EGFP:s, and were always fewer than the D^{d}-EGFP molecules. In the Ly49A-D^{d}-EGFP sample (FIG. 8, right column), the cross-correlation curve is not very high at this concentration ratio, indicating a low fraction of cis-associated Ly49A receptors. In the middle row, measurement results from cells having around four D^{d}-EGFP ligands per antibody are shown. A cross-correlation amplitude is observed for both the positive control and the Ly49A sample (FIG. 8, left and right column, respectively). The amplitude is slightly lower in the Ly49A sample, indicating that not all Ly49A are cis-associated. Also in the lowest row, displaying correlation curves from cells having around 20 D^{d}-EGFP ligands per antibody, there is a clear cross-correlation for the positive control and the Ly49A sample. In this case there is virtually no difference between the Ly49A sample and the positive control. Hence, most Ly49A can be expected to be bound in cis.

For the K^{b}-ab (the negative control), only a very limited cross-correlation was observed under these three concentration, indicating a very small cross-talk.

Thus, by visual inspection of the recorded auto- and cross-correlation curves it can be concluded that a specific cis interaction between H-2D^{d} and Ly49A can be unambiguously detected. Further, the extent of cis-interaction between Ly49A and H-2D^{d} shows a variation with the local concentrations of the species. This variation can be further analysed in a quantitative fashion. However, to perform such analyses a more detailed characterization of the FCCS instrument parameters is required. In particular, an influence from a displacement of the foci of the two lasers, and cross-talk between the two fluorescent detection channels, in particular when the concentration of D^{d}-EGFP was much higher than that of the Al647-ab, could not be excluded and thus needed to be quantified.

### Determination of Parameters Relevant for the Quantification of Cis Interactions

The above-mentioned cross-talk and the inevitable non-perfect overlap between the two excitation laser foci are acknowledged limitations of the FCCS technique, which in general make the quantification of specific interactions difficult. In principle, the influence of cross-talk and focal displacement can be taken into account in the analysis, as stated in the refined FCCS model of Eq. 4C. However, to take advantage of this model, the displacement parameter r₀ and the cross-talk parameter K have to be known. Instead of adding complex techniques to determine the displacement or use approximate methods to determine the influence of cross-talk, we took advantage of an entirely cell-based assay to find these two parameters.

### Determination of the Cross-Talk Parameter

Cross-talk between the two fluorescent detection channels gives rise to increased apparent cross-correlation amplitude. In our study the cross-talk parameter, K, is given by the ratio between the brightness of the D^{d}-EGFP and the Al647-labelled antibody, as detected in the red channel for both species. It could be directly determined by fitting Eq. 4C to the auto- and cross-correlation curves from the negative control, assuming that no reactions occur between the red and the green molecules. With the excitation intensities used in this study, fluorescence saturation can be neglected. Wₘₐₓ^{G}κ_{g}^{R}q_{g} and Wₘₐₓ^{R}κᵣ^{R}q^{r} and hence also K, are then proportional to the powers of the two lasers. The resulting cross-talk parameter, for a power ratio of one into the objective, was 0.5%+/−0.7% (20 cells) for the K^{b}-ab. By knowing the relative brightness differences between the different antibodies, K could be determined also for the positive control and the cis interaction measurements. The crosstalk parameters per power ratio unit were determined to 0.2%+/−0.2% and 0.1%+/−0.1% for the two differently labelled D^{d}-ab:s and 0.1%+/−0.2% for the Ly49-ab. Thus, the cross-talk in this study was relatively small. Apart from the fact that the emission spectra of EGFP and Al647 lies far apart from each other, a contributing reason for this low cross-talk is that each antibody contained several bright Al-647 fluorophores, while the D^{d}-EGFP only contained one EGFP.

### Determination of the Displacement Between the Centers of the Focal Areas

In dual laser FCCS measurements it is technically difficult to align the two lasers perfectly and a non-perfect overlap between the two detection areas is inevitable. As the distance between the laser foci increases, the amplitude of the cross-correlation will decrease. In this study, the positive control provides a means to estimate the displacement between the laser foci, as all red antibodies present are expected to bind specifically to D^{d}-EGFP. Hence cᵣ=0 in Eq. 4C. By fitting Eq. 5 to the experimental auto- and cross-correlation curves in the positive control with parameters set according to section 3.6, the average value of the displacement r₀ for each measurement day was determined. From these fits, also corresponding values for ω_{G} and ω_{R} could be directly determined. These average values were used in the further analysis of the cis-interaction between D^{d}-EGFP and Ly49A. The average values over all measurement days (n=35) was: 152±47 nm (standard deviation) for r₀, 222±51 nm for ω_{G}, and 270±52 nm for ω_{R}. The most obvious reason for using the radii of the effective areas determined from the cell surface experiments, rather than the radii determined from solution measurements, is that the adjustment procedure does not necessarily place the membrane where the diameters of the laser beams are the smallest.

The determined parameter value of r₀ represents an upper limit, since also other factors could give rise to decreased cross-correlation amplitudes in the positive control measurements. In particular, if a significant fraction of the antibodies were either bound to non-fluorescing D^{d}-EGFP molecules, or would bind unspecifically to some other antigen on the cell surface, decreased cross-correlation amplitudes would also be observed. However, we applied a measurement strategy where the same cells and EGFP constructs were used for both controls and test samples, and provided that the unspecific binding properties of the different antibodies do not significantly differ from each other, such potential factors should have influenced both controls and test samples equally.

### 6: Experimental Example

### Characterizing the Binding Strength of Two Interacting Species in the Membrane of Live Cells

### 4.4 Quantifying the Cis Interaction Between Ly49A and D^{d}-EGFP

Having determined the cross-talk and displacement parameters as in Experimental Example under section 5 above, it was possible to more quantitatively determine the concentrations of Ly49A and D^{d}-EGFP molecules and the fraction, γ, of cis-associated Ly49A. In total, FCCS measurements were performed on 49 cells displaying a range of different concentrations of Ly49A and D^{d}-EGFP. The FCCS data was subsequently analysed as described above.

The fraction of Ly49A receptors bound in cis was found to vary with the concentration of D^{d}-EGFP, which is characteristic for a diffusion limited bimolecular reaction. For a diffusion-limited bimolecular reaction between two species A and B in solution the equilibrium constant is defined as:

$${K}_{D} = \frac{⌊ {A}_{free} ⌋ \cdot ⌊ {B}_{free} ⌋}{[ AB ]} . \qquad ( 5 )$$

and the fraction, γ, of bound A molecules is given by:

$$γ = \frac{[ AB ]}{[ A ]} = \frac{{K}_{D} + [ A ] + [ B ] + \sqrt{{( {K}_{D} + [ A ] + [ B ] )}^{2} - 4 \cdot [ A ] \cdot [ B ]}}{2 [ A ]} . \qquad ( 6 )$$

Here, [A]=└A_{free}┘+[AB] and [B]=└B_{free}┘+[AB] are the total concentrations of A and B, respectively, with the index free denoting non-bound species. By fitting Eq. 6 to the experimentally determined parameters γ, [A]=[Ly49A], and [B]=[D^{d}-EGFP] for each of the 49 cells, K_{D} could be estimated to 45±6 molecules/μm². A similar value has been determined for ternary cytokine-receptor complexes tethered on artificial membranes.

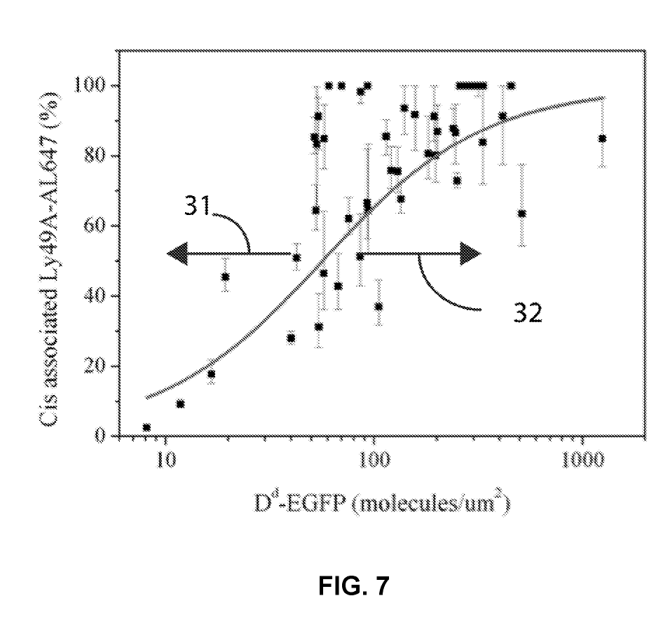

In FIG. 7, the fraction of Ly49A receptors bound in cis is plotted versus the D^{d}-EGFP concentration, regardless of the Ly49A concentration. In the cells studied, the D^{d}-EGFP concentration varied much more than the Ly49A concentration. As a first approximation, the inset can therefore be regarded as a binding plot for the average Ly49A concentration. With a larger span of Ly49A concentrations, the measured γ values would be expected to show a larger spread.

The solid line represents the binding curve when the previously determined K_{D} and the average Ly49A concentration are inserted into Eq. 6. The curve reasonably resembles a bimolecular binding curve, as predicted by Eq. 6. However, the determined K_{D}=45 μm⁻² only represents an average K_{D} within the range of Ly49 concentrations displayed by the cells in this study. Secondly, the definition of K_{D} in Eq. 5 relies on that the frequency of collisions between the two reacting species is linearly dependent on the concentrations of these species. This is typically valid for reactions occurring in three dimensions, as in a solution, but is not necessarily true for a reaction confined to the two-dimensional system of a membrane. Hence, Eq. 6 should be regarded as an approximate model describing the dependency of γ on [D^{d}-EGFP] and [Ly49A] for the cis interactions taking place in a cellular membrane.

Apart from variations in [Ly49A], the spread in γ can also be due to other biological variations between the cells, for instance in the metabolic state of the cells, or in the overall amount of proteins in the cell membranes (which in turn may influence the diffusion coefficients of the ligand and receptor molecules, see below). Nonetheless, on a cell population level and according to our analysis, many cells had close to 100% of their receptors bound in cis at lower D^{d}-EGFP concentrations than would be suggested from the fit in FIG. 7. Presuming that the cis interaction is regulated by a diffusion-driven process, this probably reflects that the cis interaction is facilitated when the diffusion of the reactants is confined to a two-dimensional reservoir. At least within a certain concentration interval, this can render the amount of Ly49 receptors bound in cis even more strongly dependent on the local D^{d} concentration, than would be expected in solution experiments. The error in the estimated concentrations is mainly due to the 20% uncertainty in ω_{G} and ω_{R}. Also bleaching is expected to have some influence on the estimated concentrations. Although no significant decay in the fluorescence intensity was observed during measurements, cumulative effects could still be prominent during the measurement times. Based on the low excitation power and the size of the cells (diameters of 5 to 10 μm), we estimate these cumulative effects to be less than 10%. In total, the error in the absolute concentration estimation is about 40%. However, the error in the relative concentration estimations of the species is expected to be significantly lower.

Thus, these experiments show that the method of the present invention, which yields high accuracy on the concentration data, may give information on how two membrane proteins interact. In this case the combination of Fluorescent Protein (FP) fused transfected receptor and an antibody labeled endogenous receptor, were used, which advantageously facilitated generation of binding plots

### 7. Determining if a Compound Promotes or Inhibits the Interaction Between Two Membrane Protein

To many diseases there are receptor-receptor interactions in the membrane of the same cell associated. Hence by discover compounds (for example small molecules or peptides) that are able to modulate (strengthen or blocking) such interactions, these compounds could potentially become novel drugs.

As described in Example 6, the strength of a particular receptor-receptor interaction in the membrane of the same cell may be quantified, with the methods explained in Example 6, and the dissociation constant for the interaction may be extracted from the fitted curve in FIG. 7. By generating curves, as showed in FIG. 7, for a particular receptor-receptor interaction, when different modulating compounds are present in the sample, and compare the change of the extracted Kd, different compounds could be distinguished from each other based on their efficacy on the particular receptor-receptor interaction.

Thus the invention could be used to discover novel drugs in various medical fields where receptor-receptor interactions play a role in the disease mechanism.

### REFERENCES

1 Magde, D., Elson, E. & Webb, W. W. Thermodynamic Fluctuations in a Reacting System—Measurement by Fluorescence Correlation Spectroscopy. Physical Review Letters 29, 705-708 (1972).
2 Elliot L. Elson, D. M. Fluorescence correlation spectroscopy. I. Conceptual basis and theory. Biopolymers 13, 1-27 (1974).
3 Eigen, M. & Kustin, K. The Kinetics of Halogen Hydrolysis. J. Am. Chem. Soc. 84, 1355-1361 (1962).
4 Schwille, P., Meyer-Almes, F. J. & Rigler, R. Dual-color fluorescence cross-correlation spectroscopy for multicomponent diffusional analysis in solution. Biophys J 72, 1878-1886 (1997).
5 Förster, T. Zwischenmolekulare Energiewanderung and Fluoreszenz. Annalen der Physik 437, 55-75 (1948).
6 Schwille, P., MeyerAlmes, F. J. & Rigler, R. Dual-color fluorescence cross-correlation spectroscopy for multicomponent diffusional analysis in solution. Biophysical Journal 72, 1878-1886 (1997).

1.-45. (canceled)

46. A FCCS method for determining the concentration and/or the diffusion coefficient of at least a first labeled species, a second labeled species and/or a complex between said first and second labeled species, in a system, with a FCCS apparatus comprising a first laser for exciting the first labeled species, the same or a second laser for exciting the second labeled species as well as a first channel for detecting fluorescence from the first labeled species and a second channel for detecting fluorescence from the second labeled species, wherein the method comprises the steps of
a) determining a cross-talk parameter K, wherein K is the ratio between the brightness of the first labeled species and the second labeled species at the centre of each focus, as detected for both species in the channel for detecting the second labeled species;
b) optionally determining a displacement parameter rₒ, wherein r₀ is the displacement between the two lasers of the FCCS apparatus in the lateral dimension if a second laser is used for exciting the second labeled species, and
c) using K, rₒ or both K and rₒ for determining the concentration and/or the diffusion coefficient of said first and/or a second labeled species and/or a complex between said first and second labeled species.

47. A method according to claim 46, further comprising the initial step a₀) of providing at least one sample comprising said system to be analysed in the FCCS apparatus and measuring the cross correlation function G_{GR}(τ) of said first and second labeled species and the autocorrelation functions, G_{G}(τ) and G_{R}(τ), of said first and second labeled species.

48. A method according to claim 47, wherein step a) and/or b) comprises fitting the autocorrelation function and the cross correlation functions obtained in step a₀ to Equations 4c:
$$\{ \begin{matrix}{G}_{GR} ( τ ) - 1 = \frac{\frac{{V}_{G} {V}_{R}}{{V}_{GR}} {c}_{gr} {e}^{- ( \begin{matrix}\frac{2 {r}_{0}^{2}}{{ω}_{G}^{2} + {ω}_{R}^{2} + 8 {D}_{gr} τ} + \\ \frac{2 {z}_{0}^{2}}{{z}_{G}^{2} + {z}_{R}^{2} + 8 {D}_{gr} τ}\end{matrix} )} {Diff}_{gr}^{GR} ( τ ) + {V}_{G} K ( {c}_{g} {Diff}_{g}^{G} ( τ ) + {c}_{gr} {Diff}_{gr}^{G} ( τ ) )}{( {V}_{G} ( {c}_{g} + {c}_{gr} ) + \frac{{bg}_{G}}{{W}_{max}^{G} {κ}_{g}^{G} {q}_{g}} ) ( {V}_{R} ( {c}_{r} + {c}_{gr} ) + \frac{{bg}_{G}}{{W}_{max}^{G} {κ}_{r}^{R} {q}_{r}} + {V}_{G} K ( {c}_{g} + {c}_{gr} ) )} \\ {G}_{R} ( τ ) - 1 = \frac{{V}_{R} ( \begin{matrix}{c}_{r} {Diff}_{r}^{R} ( τ ) + \\ {c}_{gr} {Diff}_{gr}^{R} ( τ )\end{matrix} ) + 2 \frac{{V}_{G} {V}_{R}}{{V}_{GR}} {Kc}_{gr} {e}^{- ( \begin{matrix}\frac{2 {r}_{0}^{2}}{{ω}_{G}^{2} + {ω}_{R}^{2} + 8 {D}_{gr} τ} + \\ \frac{2 {z}_{0}^{2}}{{z}_{G}^{2} + {z}_{R}^{2} + 8 {D}_{gr} τ}\end{matrix} )} {Diff}_{gr}^{GR} ( τ ) + {V}_{G} {K}^{2} ( \begin{matrix}{c}_{g} {Diff}_{g}^{G} ( τ ) + \\ {c}_{gr} {Diff}_{gr}^{G} ( τ )\end{matrix} )}{{( {V}_{R} ( {c}_{r} + {c}_{gr} ) + \frac{{bg}_{R}}{{W}_{max}^{R} {κ}_{r}^{R} {q}_{r}} + {V}_{G} K ( {c}_{g} + {c}_{gr} ) )}^{2}} \\ {G}_{G} ( τ ) - 1 = \frac{{V}_{G} ( {c}_{g} {Diff}_{g}^{G} ( τ ) + {c}_{gr} {Diff}_{gr}^{G} ( τ ) )}{{( {V}_{G} ( {c}_{g} + {c}_{gr} ) + \frac{{bg}_{G}}{{W}_{max}^{G} {κ}_{g}^{G} {q}_{g}} )}^{2}}\end{matrix} \qquad ( 4 C )$$
wherein
c_{g}, cᵣ and c_{gr} are the concentrations of the two free species and their complex, respectively;
the subscript u denotes the first species (g), the second species (r) or the their complex (gr), and Dᵤ denotes their corresponding diffusion coefficients;
bg_{G} and bg_{R} are the background fluorescence in the first and the second channel, respectively, when both lasers are on;
κ_{g}^{R} refers to the detection efficiency of the first labeled species in the detector intended to detect the second labeled species;
κᵣ^{R} refers to the detection efficiency of the second labeled species in the detector intended to detect the second labeled species;
q_{g} refers to the quantum yield of the first labeled species;
qᵣ refers to the quantum yield of the second labeled species;
V_{G} and V_{R} are the effective detection volumes of the green and red laser foci, and V_{GR} is the corresponding green-red detection volume;
W( r̅)=CEF( r̅)I_{exc}( r̅) is the detected fluorescence brightness distribution, a product of the excitation intensity I_{exc}( r̅) and the collection efficiency function CEF( r̅);
the radial distances from the maximum point of W_{G}( r̅) and W_{R}( r̅) to where they have dropped by a factor of e² is denoted ω_{G} and ω_{R} in the lateral direction and z_{G} and z_{R} in the axial direction, respectively;
Wₘₐₓ^{G} refers to the maximal value of the brightness distribution, W_{G}( r̅) of the first labeled species;
Wₘₐₓ^{R} refers to the maximal value of the brightness distribution, W_{R}( r̅), of the second labeled species; and
$$\{ \begin{matrix}{Diff}_{u}^{G} ( τ ) = {( 1 + \frac{4 {D}_{u} τ}{{ω}_{G}^{2}} )}^{- 1} {( 1 + \frac{4 {D}_{u} τ}{{z}_{G}^{2}} )}^{- 1 / 2} \\ {Diff}_{u}^{R} ( τ ) = {( 1 + \frac{4 {D}_{u} τ}{{ω}_{R}^{2}} )}^{- 1} {( 1 + \frac{4 {D}_{u} τ}{{z}_{R}^{2}} )}^{- 1 / 2} \\ {Diff}_{gr}^{GR} ( τ ) = {( 1 + \frac{4 {D}_{gr} τ}{( {ω}_{G}^{2} + {ω}_{R}^{2} ) / 2} )}^{- 1} {( 1 + \frac{4 {D}_{gr} τ}{( {z}_{G}^{2} + {z}_{R}^{2} ) / 2} )}^{- 1 / 2}\end{matrix} ;$$
and wherein step c) comprises fitting the autocorrelation functions and the cross correlation function and using the determined K and/or rₒ in Equations 4c for determining the concentration and/or the diffusion coefficient of said first and/or a second labeled species and/or a complex between said first and second labeled species.

49. A method according to claim 48, comprising the step of globally fitting the autocorrelation curves and the cross correlation curve.

50. A method according to claim 46, wherein step b) involves determining the displacement parameter r₀ using the cross-talk parameter K.

51. A method for calculating the displacement r₀ between the excitation foci of two lasers, comprising performing FCCS measurements on two species that interact with each other (a positive control); and further comprising the steps of
a) determining a cross-talk parameter K, wherein K is the ratio between the brightness of a first labeled species and a second labeled species at the centre of each focus, as detected for both species in a channel for detecting the second labeled species;
b) using the positive control and/or the cross talk parameter K for determining the displacement parameter rₒ.

52. A method according to claim 51, further comprising the initial step a₀) of providing at least one sample comprising a first labeled species and a second labeled species to be analysed in the FCCS apparatus and measuring the cross correlation function G_{GR}(τ) of said first and second labeled species and the autocorrelation functions, G_{G}(τ) and G_{R}(τ), of said first and second labeled species in said sample.

53. A method according to claim 52, wherein step a) and/or b) comprises fitting the autocorrelation function and the cross correlation functions obtained in step a₀ to Equations 4c:
$$\{ \begin{matrix}{G}_{GR} ( τ ) - 1 = \frac{\frac{{V}_{G} {V}_{R}}{{V}_{GR}} {c}_{gr} {e}^{- ( \begin{matrix}\frac{2 {r}_{0}^{2}}{{ω}_{G}^{2} + {ω}_{R}^{2} + 8 {D}_{gr} τ} + \\ \frac{2 {z}_{0}^{2}}{{z}_{G}^{2} + {z}_{R}^{2} + 8 {D}_{gr} τ}\end{matrix} )} {Diff}_{gr}^{GR} ( τ ) + {V}_{G} K ( {c}_{g} {Diff}_{g}^{G} ( τ ) + {c}_{gr} {Diff}_{gr}^{G} ( τ ) )}{( {V}_{G} ( {c}_{g} + {c}_{gr} ) + \frac{{bg}_{G}}{{W}_{max}^{G} {κ}_{g}^{G} {q}_{g}} ) ( {V}_{R} ( {c}_{r} + {c}_{gr} ) + \frac{{bg}_{G}}{{W}_{max}^{G} {κ}_{r}^{R} {q}_{r}} + {V}_{G} K ( {c}_{g} + {c}_{gr} ) )} \\ {G}_{R} ( τ ) - 1 = \frac{{V}_{R} ( \begin{matrix}{c}_{r} {Diff}_{r}^{R} ( τ ) + \\ {c}_{gr} {Diff}_{gr}^{R} ( τ )\end{matrix} ) + 2 \frac{{V}_{G} {V}_{R}}{{V}_{GR}} {Kc}_{gr} {e}^{- ( \begin{matrix}\frac{2 {r}_{0}^{2}}{{ω}_{G}^{2} + {ω}_{R}^{2} + 8 {D}_{gr} τ} + \\ \frac{2 {z}_{0}^{2}}{{z}_{G}^{2} + {z}_{R}^{2} + 8 {D}_{gr} τ}\end{matrix} )} {Diff}_{gr}^{GR} ( τ ) + {V}_{G} {K}^{2} ( \begin{matrix}{c}_{g} {Diff}_{g}^{G} ( τ ) + \\ {c}_{gr} {Diff}_{gr}^{G} ( τ )\end{matrix} )}{{( {V}_{R} ( {c}_{r} + {c}_{gr} ) + \frac{{bg}_{R}}{{W}_{max}^{R} {κ}_{r}^{R} {q}_{r}} + {V}_{G} K ( {c}_{g} + {c}_{gr} ) )}^{2}} \\ {G}_{G} ( τ ) - 1 = \frac{{V}_{G} ( {c}_{g} {Diff}_{g}^{G} ( τ ) + {c}_{gr} {Diff}_{gr}^{G} ( τ ) )}{{( {V}_{G} ( {c}_{g} + {c}_{gr} ) + \frac{{bg}_{G}}{{W}_{max}^{G} {κ}_{g}^{G} {q}_{g}} )}^{2}}\end{matrix} \qquad ( 4 C )$$
wherein
c_{g}, cᵣ and C_{gr} are the concentrations of the two free species and their complex, respectively;
the subscript u denotes the first species (g), the second species (r) or the their complex (gr), and Dᵤ denotes their corresponding diffusion coefficients;
bg_{G} and bg_{R} are the background fluorescence in the first and the second channel, respectively, when both lasers are on;
κ_{d}^{R} refers to the detection efficiency of the first labeled species in the detector intended to detect the second labeled species;
κᵣ^{R} refers to the detection efficiency of the second labeled species in the detector intended to detect the second labeled species;
q_{g} refers to the quantum yield of the first labeled species;
qᵣ refers to the quantum yield of the second labeled species;
V_{G} and V_{R} are the effective detection volumes of the green and red laser foci, and V_{GR} is the corresponding green-red detection volume;
W( r̅)=CEF( r̅)I_{exc}( r̅) is the detected fluorescence brightness distribution, a product of the excitation intensity I_{exc}( r̅) and the collection efficiency function CEF( r̅);
the radial distances from the maximum point of W_{G}( r̅) and W_{R}( r̅) to where they have dropped by a factor of e² is denoted ω_{G} and ω_{R} in the lateral direction and z_{G} and z_{R} in the axial direction, respectively;
Wₘₐₓ^{G} refers to the maximal value of the brightness distribution, W_{G}( r̅) of the first labeled species;
Wₘₐₓ^{R} refers to the maximal value of the brightness distribution, W_{R}( r̅), of the second labeled species; and
$$\{ \begin{matrix}{Diff}_{u}^{G} ( τ ) = {( 1 + \frac{4 {D}_{u} τ}{{ω}_{G}^{2}} )}^{- 1} {( 1 + \frac{4 {D}_{u} τ}{{z}_{G}^{2}} )}^{- 1 / 2} \\ {Diff}_{u}^{R} ( τ ) = {( 1 + \frac{4 {D}_{u} τ}{{ω}_{R}^{2}} )}^{- 1} {( 1 + \frac{4 {D}_{u} τ}{{z}_{R}^{2}} )}^{- 1 / 2} \\ {Diff}_{gr}^{GR} ( τ ) = {( 1 + \frac{4 {D}_{gr} τ}{( {ω}_{G}^{2} + {ω}_{R}^{2} ) / 2} )}^{- 1} {( 1 + \frac{4 {D}_{gr} τ}{( {z}_{G}^{2} + {z}_{R}^{2} ) / 2} )}^{- 1 / 2}\end{matrix} .$$

54. A method according to claim 53, comprising the step of globally fitting the autocorrelation curves and the cross correlation curve.

55. A method according to claim 51, wherein the first and second species are labeled DNA strands.

56. A method according to claim 51, wherein the system is a single cell, and wherein the first species is a labeled binding agent and the second species is a labeled membrane protein, or vice versa.

57. A method according to claim 46, wherein the fluorescence emission of the first labeled species is blue shifted with respect to the fluorescence emission from the second species, and the channels for detecting each of the labeled species are suitable for their respective spectral range of their fluorescence.

58. A method according to claim 46, wherein K is defined as
$$K = \frac{{W}_{max}^{G} {κ}_{g}^{R} {q}_{g} {σ}_{g}}{{W}_{max}^{R} {κ}_{r}^{R} {q}_{r} {σ}_{r}}$$
wherein
σ_{g} is the excitation cross section of the first labeled species;
σᵣ is the excitation cross section of the second labeled species;
Wₘₐₓ^{G} refers to the maximal value of the brightness distribution, W_{G}( r̅) of the first labeled species;
κ_{g}^{R} refers to the detection efficiency of the first labeled species in the detector intended to detect the second labeled species;
q_{g} refers to the quantum yield of the first labeled species;
Wₘₐₓ^{R} refers to the maximal value of the brightness distribution, W_{R}( r̅), of the second labeled species;
κᵣ^{R} refers to the detection efficiency of the second labeled species in the detector intended to detect the second labeled species; and
qᵣ refers to the quantum yield of the second labeled species.

59. A method according to claim 46, wherein the step of determining K comprises determining K via a negative control, in which two species lacking mutual interactions are utilized.

60. A method according to claim 46, wherein the step of determining r₀ comprises performing FCCS measurements on two species that interact with each other (a positive control).

61. A FCCS device for determining the concentration and/or the diffusion coefficient of at least a first labeled species, a second labeled species and/or a complex between said first and second labeled species, said device comprising:
a FCCS apparatus comprising a first laser for exciting the first labeled species, the same or a second laser for exciting the second labeled species, a first channel for detecting fluorescence from the first labeled species and a second channel for detecting fluorescence from the second labeled species; and
an estimation unit adapted to:
determine a cross-talk parameter K, wherein K is the ratio between the brightness of the first labeled species and the second labeled species at the centre of each focus, as detected for both species in the channel for detecting the second labeled species;
determine a displacement parameter rₒ, wherein r₀ is the displacement between the two lasers of the FCCS apparatus in the lateral dimension if a second laser is used for exciting the second labeled species, and
determine the concentration and/or the diffusion coefficient of said first and/or a second labeled species by the use of the determined K, r₀ or both the determined K and rₒ.

62. A FCCS device according to claim 61, wherein the estimation unit is further adapted to measure the cross correlation function G_{GR}(τ) of said first and second labeled species and the autocorrelation functions, G_{G}(τ) and G_{R}(τ), of said first and second labeled species

63. A FCCS device according to claim 62, wherein the estimation unit is further adapted to fit the autocorrelation function and the cross correlation functions obtained to Equations 4c:
$$\{ \begin{matrix}{G}_{GR} ( τ ) - 1 = \frac{\frac{{V}_{G} {V}_{R}}{{V}_{GR}} {c}_{gr} {e}^{- ( \begin{matrix}\frac{2 {r}_{0}^{2}}{{ω}_{G}^{2} + {ω}_{R}^{2} + 8 {D}_{gr} τ} + \\ \frac{2 {z}_{0}^{2}}{{z}_{G}^{2} + {z}_{R}^{2} + 8 {D}_{gr} τ}\end{matrix} )} {Diff}_{gr}^{GR} ( τ ) + {V}_{G} K ( {c}_{g} {Diff}_{g}^{G} ( τ ) + {c}_{gr} {Diff}_{gr}^{G} ( τ ) )}{( {V}_{G} ( {c}_{g} + {c}_{gr} ) + \frac{{bg}_{G}}{{W}_{max}^{G} {κ}_{g}^{G} {q}_{g}} ) ( {V}_{R} ( {c}_{r} + {c}_{gr} ) + \frac{{bg}_{G}}{{W}_{max}^{G} {κ}_{r}^{R} {q}_{r}} + {V}_{G} K ( {c}_{g} + {c}_{gr} ) )} \\ {G}_{R} ( τ ) - 1 = \frac{{V}_{R} ( \begin{matrix}{c}_{r} {Diff}_{r}^{R} ( τ ) + \\ {c}_{gr} {Diff}_{gr}^{R} ( τ )\end{matrix} ) + 2 \frac{{V}_{G} {V}_{R}}{{V}_{GR}} {Kc}_{gr} {e}^{- ( \begin{matrix}\frac{2 {r}_{0}^{2}}{{ω}_{G}^{2} + {ω}_{R}^{2} + 8 {D}_{gr} τ} + \\ \frac{2 {z}_{0}^{2}}{{z}_{G}^{2} + {z}_{R}^{2} + 8 {D}_{gr} τ}\end{matrix} )} {Diff}_{gr}^{GR} ( τ ) + {V}_{G} {K}^{2} ( \begin{matrix}{c}_{g} {Diff}_{g}^{G} ( τ ) + \\ {c}_{gr} {Diff}_{gr}^{G} ( τ )\end{matrix} )}{{( {V}_{R} ( {c}_{r} + {c}_{gr} ) + \frac{{bg}_{R}}{{W}_{max}^{R} {κ}_{r}^{R} {q}_{r}} + {V}_{G} K ( {c}_{g} + {c}_{gr} ) )}^{2}} \\ {G}_{G} ( τ ) - 1 = \frac{{V}_{G} ( {c}_{g} {Diff}_{g}^{G} ( τ ) + {c}_{gr} {Diff}_{gr}^{G} ( τ ) )}{{( {V}_{G} ( {c}_{g} + {c}_{gr} ) + \frac{{bg}_{G}}{{W}_{max}^{G} {κ}_{g}^{G} {q}_{g}} )}^{2}}\end{matrix} \qquad ( 4 C )$$
wherein
c_{g}, cᵣ and C_{gr} are the concentrations of the two free species and their complex, respectively;
the subscript u denotes the first species (g), the second species (r) or the their complex (gr), and Dᵤ denotes their corresponding diffusion coefficients;
bg_{G} and bg_{R} are the background fluorescence in the first and the second channel, respectively, when both lasers are on;
κ_{g}^{R} refers to the detection efficiency of the first labeled species in the detector intended to detect the second labeled species;
κᵣ^{R} refers to the detection efficiency of the second labeled species in the detector intended to detect the second labeled species;
q_{g} refers to the quantum yield of the first labeled species;
qᵣ refers to the quantum yield of the second labeled species;
V_{G} and V_{R} are the effective detection volumes of the green and red laser foci, and V_{GR} is the corresponding green-red detection volume;
W( r̅)=CEF( r̅)I_{exc}( r̅) is the detected fluorescence brightness distribution, a product of the excitation intensity I_{exc}( r̅) and the collection efficiency function CEF( r̅);
the radial distances from the maximum point of W_{G}( r̅) and W_{R}( r̅) to where they have dropped by a factor of e² is denoted ω_{G} and ω_{R} in the lateral direction and z_{G} and z_{R} in the axial direction, respectively;
Wₘₐₓ^{G} refers to the maximal value of the brightness distribution, W_{G}( r̅) of the first labeled species;
Wₘₐₓ^{R} refers to the maximal value of the brightness distribution, W_{R}( r̅), of the second labeled species; and
$$\{ \begin{matrix}{Diff}_{u}^{G} ( τ ) = {( 1 + \frac{4 {D}_{u} τ}{{ω}_{G}^{2}} )}^{- 1} {( 1 + \frac{4 {D}_{u} τ}{{z}_{G}^{2}} )}^{- 1 / 2} \\ {Diff}_{u}^{R} ( τ ) = {( 1 + \frac{4 {D}_{u} τ}{{ω}_{R}^{2}} )}^{- 1} {( 1 + \frac{4 {D}_{u} τ}{{z}_{R}^{2}} )}^{- 1 / 2} \\ {Diff}_{gr}^{GR} ( τ ) = {( 1 + \frac{4 {D}_{gr} τ}{( {ω}_{G}^{2} + {ω}_{R}^{2} ) / 2} )}^{- 1} {( 1 + \frac{4 {D}_{gr} τ}{( {z}_{G}^{2} + {z}_{R}^{2} ) / 2} )}^{- 1 / 2}\end{matrix} ;$$
and to use the determined K and rₒ in Equation 4c for determining the concentration and/or the diffusion coefficient of said first and/or a second labeled species and/or a complex between said first and second labeled species.

64. A FCCS device according to claim 63, wherein the estimation unit is adapted to fit the autocorrelation curves and the cross correlation curves to Eq. 4C globally.

65. A FCCS device according to claim 61, wherein the estimation unit is adapted to determine r₀ by the use of the determined cross talk parameter K.
